(19) United States
(12) Patent Application Publication
(10) Pub. No.: US 2026/0199290 A1
(43) Pub. Date: Jul. 16, 2026

(54) FORMULATIONS OF 5-MEO-DMT

(71) Applicant: Beckley Psytech Limited, Oxford (GB)

(72) Inventor: Cosmo FEILDING-MELLEN

(21) Appl. No.: 19/129,744

(22) PCT Filed: Nov. 14, 2023
(86) PCT No.: PCT/GB2023/052977

§ 371 Date: May 14, 2025

Related U.S. Application Data
(63) Continuation-in-part of application No. 18/065,030, filed on Dec. 13, 2022, now abandoned.

(30) Foreign Application Priority Data
Nov. 14, 2022 (GB) 2216961.9

(51) Int. Cl. A61K 31/4045; A61K 9/00; A61K 9/16; A61P 25/24

(52) U.S. Cl. CPC A61K 31/4045; A61K 9/0043; A61K 9/16; A61P 25/24

(57) ABSTRACT

A pharmaceutically acceptable composition may include 5-methoxy-N,N-dimethyltryptamine (5-MeO-DMT). A method of treating a subject may include administering the composition to the subject. The method may treat or prevent depression in the subject. Administration of the composition to the subject may produce in the subject a blood plasma Cmax of about 4 ng/mL to about 39 ng/mL.

### FIELD OF THE INVENTION

This invention relates to formulations of 5-methoxy-N,N-dimethyltryptamine (5-MeO-DMT) and uses thereof.

### BACKGROUND

5-MeO-DMT benzoate is the benzoate salt of the pharmacologically active compound of the tryptamine class, 5-MeO-DMT, and has the following structure:

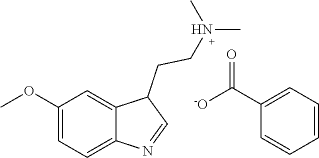

5-MeO-DMT is a psychoactive/psychedelic substance found in nature and is believed to act mainly through serotonin receptors. It is also believed to have a high affinity for the 5-HT2 and 5-HT1A subtypes, and/or inhibits monoamine reuptake. There remains a need in the art for improved formulations of 5-MeO-DMT benzoate.

### SUMMARY

It has surprisingly been discovered that the benzoate salt of 5-MeO-DMT displays dose-proportional pharmacokinetics. This is surprising as it has previously been found that the hydrochloride salt of 5-MeO-DMT displays non-dose proportional pharmacokinetics. It is desirable that a compound for use in treatment has dose-proportional pharmacokinetics, for example, to facilitate dose and dose regimen adjustment in patients. As used herein, the terms “approximately” and “about” generally should be understood to encompass ±5% of a specified amount or value. As used herein, “dose proportional” should be understood to mean that increases in the administered dose are accompanied by proportional increases in the PK profile, such as the AUC or Cmax.

In an embodiment, there is provided a dose-proportional pharmaceutically acceptable dry powder intranasal composition comprising 5-methoxy-N,N-dimethyltryptamine (5-MeO-DMT) benzoate dry powder particles, and one or more pharmaceutically acceptable carriers or excipients, for use in a method of treatment, wherein the composition has dose-proportional pharmacokinetics. In an embodiment, there is provided a pharmaceutically acceptable composition comprising 5-MeO-DMT, wherein administration of said composition to a subject produces in said subject a blood plasma Cmax (ng/mL) of about 4-39, of about 5-35 or of about 6-29. In an embodiment, administration of said composition to a subject produces in said subject a Tmax (h) of about 0.05-0.5, of about 0.06-0.3 or of about 0.1-0.25. In an embodiment, administration of said composition to a subject produces in said subject a t½ (h) of about 0.1-0.55, of about 0.2-0.5 or of about 0.25-0.44. In an embodiment, administration of said composition to a subject produces in said subject a AUClast (h*ng/mL) of about 1.0-22, of about 1.2-20 or of about 1.5-18.5. In an embodiment, administration of said composition to a subject produces in said subject a AUCinf (h*ng/mL) of about 1.5-27, of about 1.7-25 or of about 1.9-24. As used herein, Cmax may refer to the maximum concentration (e.g., maximum blood plasma concentration) of a compound, as the result of the administration of a composition comprising the compound. Tmax may refer to the time required for the concentration of the compound to reach Cmax, after the administration of the composition. T½ (e.g., half-life) may refer to time it takes for the concentration of the compound to halve (e.g., reach half of the Cmax), after the administration of the composition. AUClast may refer to an area under a curve representing plasma concentration as a function of time. The area may include the last measured plasma concentration. AUCinf may refer to an area under a curve representing plasma concentration as a function of time, extrapolated to where time approaches infinity. In an embodiment, the composition comprises 5-MeO-DMT benzoate. In an embodiment, the 5-MeO-DMT is present as the benzoate salt. In an embodiment, the 5-MeO-DMT composition is a composition for intranasal administration. In an embodiment, the 5-MeO-DMT composition is an intranasal composition. In an embodiment, the composition comprises 1-12 mg of 5-MeO-DMT benzoate. In an embodiment, the composition comprises 1 mg of 5-MeO-DMT benzoate, wherein intranasal administration of said composition to a subject produces in said subject one or more of: a Cmax (ng/mL) of about 4-8, of about 5-7 or of about 6; a Tmax (h) of about 0.05-0.2, of about 0.06-0.15 or of about 0.1; a t½ (h) of about 0.1-0.4, of about 0.2-0.3 or of about 0.25; a AUClast (h*ng/mL) of about 1.0-1.8, of about 1.2-1.6 or of about 1.5; or a AUCinf (h*ng/mL) of about 1.5-2.3, of about 1.7-2.0 or of about 1.9. In an embodiment, the composition comprises 4 mg of 5-MeO-DMT benzoate, wherein intranasal administration of said composition to a subject produces in said subject one or more of: a Cmax (ng/mL) of about 3-13, of about 7-11 or of about 9; a Tmax (h) of about 0.03-0.2, of about 0.09-0.16 or of about 0.12; a t½ (h) of about 0.27-0.53, of about 0.30-0.45 or of about 0.37; a AUClast (h*ng/mL) of about 1.9-7.2, of about 3.5-5.5 or of about 4.5; or a AUCinf (h*ng/mL) of about 2.4-7.4, of about 3.5-6.5 or of about 5. In an embodiment, the composition comprises 8 mg of 5-MeO-DMT benzoate, wherein intranasal administration of said composition to a subject produces in said subject one or more of: a Cmax (ng/mL) of about 16.4-30.9, of about 19-25 or of about 22; a Tmax (h) of about 0.1-0.27, of about 0.1-0.22 or of about 0.17; a t½ (h) of about 0.21-0.37, of about 0.25-0.35 or of about 0.30; a AUClast (h*ng/mL) of about 9.25-17.23, of about 11-15 or of about 13.1; or a AUCinf (h*ng/mL) of about 9.42-18.7, of about 11-16 or of about 13.9. In an embodiment, the composition comprises 10 mg of 5-MeO-DMT benzoate, wherein intranasal administration of said composition to a subject produces in said subject one or more of: a Cmax (ng/mL) of about 10.4-46.4, of about 25-39 or of about 32; a Tmax (h) of about 0.03-0.27, of about 0.1-0.22 or of about 0.14; a t½ (h) of about 0.24-0.59, of about 0.29-0.47 or of about 0.38; a AUClast (h*ng/mL) of about 9.37-20.41, of about 13-18 or of about 15.4; or a AUCinf (h*ng/mL) of about 9.42-18.7, of about 11-16 or of about 13.9. In an embodiment, the composition comprises 12 mg of 5-MeO-DMT benzoate, wherein intranasal administration of said composition to a subject produces in said subject one or more of: a Cmax (ng/mL) of about 20.9-39, of about 25-35 or of about 29; a Tmax (h) of about 0.17-0.5, of about 0.2-0.3 or of about 0.25; a t½ (h) of about 0.28-0.55, of about 0.40-0.50 or of about 0.44; a AUClast (h*ng/mL) of about 14.45-22, of about 16-20 or of about 18.5; or a AUCinf (h*ng/mL) of about 19.10-27, of about 22-25 or of about 23.9. In an embodiment, the composition is a dry powder composition. In an embodiment, the powder is characterised by one or more of: particles having a median diameter of less than 2000 μm, 1000 μm, 500 μm, 250 μm, 100 μm, 50 μm, or 1 μm; particles having a median diameter of less than 15, 14, 13, 12, 11, or 10 m; particles having a median diameter of less than 9 μm; particles having a median diameter of greater than 500 μm, 250 μm, 100 μm, 50 μm, 1 μm or 0.5 μm; and/or a particle size distribution of d10=20-60 μm, and/or d50=80-120 μm, and/or d90=130-300 μm.

In an embodiment, the composition comprises one or more pharmaceutically acceptable carriers or excipients. In an embodiment, the composition comprises one or more of: HPMC, carbomers, xanthan gum, carrageenan, copolymers of methyl vinyl ether and maleic anhydride (PVM/MA), hydroxypropyl cellulose (HPC) or sodium carboxymethylcellulose (Na-CMC). In an embodiment, the composition comprises one or more of chitosan, chitosan derivatives (such as N,N,N-trimethyl chitosan (TMC), n-propyl-(QuatPropyl), n-butyl-(QuatButyl) and n-hexyl (QuatHexyl)-N,N-dimethyl chitosan, chitosan chloride), β-cyclodextrin, Clostridium perfringens enterotoxin, zonula occludens toxin (ZOT), human neutrophil elastase inhibitor (ER143), sodium taurocholate, sodium deoxycholate sodium, sodium lauryl sulphate, glycodeoxycholat, palmitic acid, palmitoleic acid, stearic acid, oleyl acid, oleyl alcohol, capric acid sodium salt, DHA, EPA, dipalmitoyl phophatidyl choline, soybean lecithin, lysophosphatidylcholine, dodecyl maltoside, tetradecyl maltoside, EDTA, lactose, cellulose, and citric acid.

In an embodiment, the 5-MeO-DMT is present as crystalline 5-MeO-DMT benzoate as characterised by peaks in an XRPD diffractogram at 17.5, 17.7 and 21.0° 2θ±0.1° 2θ.

In an embodiment, there is provided a pharmaceutically acceptable dry powder intranasal composition comprising 1-12 mg of 5-MeO-DMT benzoate and HPMC.

In an embodiment, intranasal administration of the composition to a subject produces in said subject one or more of: a Cmax (ng/mL) of about 4-39, of about 5-35 or of about 6-29; a Tmax (h) of about 0.05-0.5, of about 0.06-0.3 or of about 0.1-0.25; a t½ (h) of about 0.1-0.55, of about 0.2-0.5 or of about 0.25-0.44; a AUClast (h*ng/mL) of about 1.0-22, of about 1.2-20 or of about 1.5-18.5; or a AUCinf (h*ng/mL) of about 1.5-27, of about 1.7-25 or of about 1.9-23.9.

In an embodiment, there is provided the use of the composition for the treatment or prevention of a disease or condition. In an embodiment, the disease or condition is depression or treatment-resistant depression. Herein disclosed, there is provided a formulation of 5-MeO-DMT benzoate. In an embodiment, there is provided a pharmaceutically acceptable composition comprising 5-MeO-DMT (hereafter ‘5-MeO-DMT composition’) comprising 1 mg 5-MeO-DMT benzoate wherein intranasal administration of said composition to a subject produces in said subject one or more of: a Cmax (ng/mL) of about 4-8, of about 5-7 or about 6; a Tmax (h) of about 0.05-0.2, of about 0.06-0.15 or about 0.1; a t½ (h) of about 0.1-0.4, of about 0.2-0.3 or about 0.25; a AUClast (h*ng/mL) of about 1.0-1.8, of about 1.2-1.6 or about 1.5; or a AUCinf (h*ng/mL) of about 1.5-2.3, of about 1.7-2.0 or about 1.9.

All pharmacokinetic values refer to, for example, the Cmax in blood plasma. In an embodiment, there is provided a 5-MeO-DMT composition comprising 2.5 mg 5-MeO-DMT benzoate wherein intranasal administration of said composition to a subject produces in said subject one or more of: a Cmax (ng/mL) of about 4-12, of about 6-10 or about 8; a Tmax (h) of about 0.1-0.3, of about 0.15-0.25 or about 0.18; t½ (h) of about 0.1-0.4, of about 0.20-0.35 or about 0.32; a AUClast (h*ng/mL) of about 2.3-6.5, of about 3.0-4.4 or about 3.8; or a AUCinf (h*ng/mL) of about 2.9-6.8, of about 3.5-5.5 or about 4.4.

In an embodiment, there is provided a 5-MeO-DMT composition comprising 4 mg 5-MeO-DMT benzoate wherein intranasal administration of said composition to a subject produces in said subject one or more of: a Cmax (ng/mL) of about 3-13, of about 7-11 or about 9; a Tmax (h) of about 0.03-0.2, of about 0.09-0.16 or about 0.12; a t½ (h) of about 0.27-0.53, of about 0.30-0.45 or about 0.37; a AUClast (h*ng/mL) of about 1.9-7.2, of about 3.5-5.5 or about 4.5; or a AUCinf (h*ng/mL) of about 2.4-7.4, of about 3.5-6.5 or about 5.

In an embodiment, there is provided a 5-MeO-DMT composition comprising 6 mg 5-MeO-DMT benzoate wherein intranasal administration of said composition to a subject produces in said subject one or more of: a Cmax (ng/mL) of about 7.7-19.3, of about 12.5-17 or about 15; a Tmax (h) of about 0.07-0.27, of about 0.1-0.2 or about 0.15; a t½ (h) of about 0.32-0.42, of about 0.35-0.4 or about 0.37; a AUClast (h*ng/mL) of about 4.6-12, of about 6-10 or about 8; or a AUCinf (h*ng/mL) of about 8.6-11, of about 9-10.5 or about 9.8.

In an embodiment, there is provided a 5-MeO-DMT composition comprising 8 mg 5-MeO-DMT benzoate wherein intranasal administration of said composition to a subject produces in said subject one or more of: a Cmax (ng/mL) of about 16.4-30.9, of about 19-25 or about 22; a Tmax (h) of about 0.1-0.27, of about 0.1-0.22 or about 0.17; a t½ (h) of about 0.21-0.37, of about 0.25-0.35 or about 0.30; a AUClast (h*ng/mL) of about 9.25-17.23, of about 11-15 or about 13.1; or a AUCinf (h*ng/mL) of about 9.42-18.7, of about 11-16 or about 13.9.

In an embodiment, the composition comprises 1-20 mg of 5-MeO-DMT, or a pharmaceutically acceptable salt thereof. In an embodiment, there is provided a 5-MeO-DMT composition comprising 10 mg 5-MeO-DMT benzoate wherein intranasal administration of said composition to a subject produces in said subject one or more of: a Cmax (ng/mL) of about 10.4-46.4, of about 25-39 or about 32; a Tmax (h) of about 0.03-0.27, of about 0.1-0.22 or about 0.14; a t½ (h) of about 0.24-0.59, of about 0.29-0.47 or about 0.38; a AUClast (h*ng/mL) of about 9.37-20.41, of about 13-18 or about 15.4; or a AUCinf (h*ng/mL) of about 9.42-18.7, of about 11-16 or about 13.9.

In an embodiment, there is provided a 5-MeO-DMT composition comprising 12 mg 5-MeO-DMT benzoate wherein intranasal administration of said composition to a subject produces in said subject one or more of: a Cmax (ng/mL) of about 20.9-38.6, of about 25-35 or about 29; a Tmax (h) of about 0.17-0.5, of about 0.2-0.3 or about 0.25; a t½ (h) of about 0.28-0.55, of about 0.40-0.50 or about 0.44; a AUClast (h*ng/mL) of about 14.45-22.23, of about 16-20 or about 18.5; or a AUCinf (h*ng/mL) of about 19.10-27.15, of about 22-25 or about 23.9.

In an embodiment, there is provided a 5-MeO-DMT composition comprising 1-12 mg 5-MeO-DMT benzoate wherein intranasal administration of said composition to a subject produces in said subject one or more of: a Cmax (ng/mL) of about 4-38.6, of about 5-35 or about 6-29; a Tmax (h) of about 0.05-0.5, of about 0.06-0.3 or about 0.1-0.25; a t½ (h) of about 0.1-0.55, of about 0.2-0.5 or about 0.25-0.44; a AUClast (h*ng/mL) of about 1.0-22.23, of about 1.2-20 or about 1.5-18.5; or a AUCinf (h*ng/mL) of about 1.5-27.15, of about 1.7-25 or about 1.9-23.9.

In an embodiment, the composition produces in a subject, per each 10 mg of 5-MeO-DMT present in the composition, one or more of: a Cmax (ng/mL) of about 10.4-46.4, of about 25-39 or about 32; a Tmax (h) of about 0.03-0.27, of about 0.1-0.22 or about 0.14; a t½ (h) of about 0.24-0.59, of about 0.29-0.47 or about 0.38; a AUClast (h*ng/mL) of about 9.37-20.41, of about 13-18 or about 15.4; or a AUCinf (h*ng/mL) of about 9.42-18.7, of about 11-16 or about 13.9.

In an embodiment, the 5-MeO-DMT composition comprises a mucoadhesive. In an embodiment, the 5-MeO-DMT composition comprises one or more of: HPMC, carbomers, xanthan gum, carrageenan, copolymers of methyl vinyl ether and maleic anhydride (PVM/MA), hydroxypropyl cellulose (HPC) or sodium carboxymethylcellulose (Na-CMC). In an embodiment, the 5-MeO-DMT composition comprises HPMC. In an embodiment, the 5-MeO-DMT composition comprises crystalline 5-MeO-DMT benzoate, as described subsequently or previously. In an embodiment the composition disclosed herein is for use as a medicament. In an embodiment, there is provided use of a composition as described previously or subsequently for the treatment of a disease or condition.

In an embodiment, the disease or condition is depression. In an embodiment, the disease or condition is treatment-resistant depression. In an embodiment, the disease or condition is: conditions caused by dysfunctions of the central nervous system, conditions caused by dysfunctions of the peripheral nervous system, conditions benefiting from sleep regulation (such as insomnia), conditions benefiting from analgesics (such as chronic pain), migraines, trigeminal autonomic cephalgias (such as short-lasting unilateral neuralgiform headache with conjunctival injection and tearing (SUNCT), and short-lasting neuralgiform headaches with cranial autonomic symptoms (SUNA)), conditions benefiting from neurogenesis (such as stroke, traumatic brain injury, Parkinson's dementia), conditions benefiting from anti-inflammatory treatment, depression, treatment resistant depression, anxiety, substance use disorder, addictive disorder, gambling disorder, eating disorders, obsessive-compulsive disorders, or body dysmorphic disorders, optionally the condition is SUNCT and/or SUNA, alcohol-related diseases and disorders, eating disorders, impulse control disorders, nicotine-related disorders, tobacco-related disorders, methamphetamine-related disorders, amphetamine-related disorders, cannabis-related disorders, cocaine-related disorders, hallucinogen use disorders, inhalant-related disorders, benzodiazepine abuse or dependence related disorders, opioid-related disorders, tobacco addiction, alcohol abuse and/or addiction.

In an embodiment there is provided a method of use of the composition disclosed herein. In an embodiment, the method of use is a method of treatment. In an embodiment the method of treatment is a method of treatment of more than one of the above conditions, for example, the method of treatment may be a method of treatment of depression and anxiety. In an embodiment the composition is administered one or more times a year. In an embodiment the composition is administered one or more times a month. In an embodiment the composition is administered one or more times a week. In an embodiment the composition is administered one or more times a day. In an embodiment the composition is administered at such a frequency as to avoid tachyphylaxis. In an embodiment the composition is administered together with a complementary treatment and/or with a further active agent. In an embodiment the further active agent is a psychedelic compound, optionally a tryptamine. In an embodiment the further active agent is lysergic acid diethylamide (LSD), psilocybin, psilocin or a prodrug thereof. In an embodiment the further active agent is an antidepressant compound. In an embodiment the further active agent is selected from an SSRI, SNRI, TCA or other antidepressant compounds.

In an embodiment the further active agent is selected from Citalopram (Celexa, Cipramil), Escitalopram (Lexapro, Cipralex), Fluoxetine (Prozac, Sarafem), Fluvoxamine (Luvox, Faverin), Paroxetine (Paxil, Seroxat), Sertraline (Zoloft, Lustral), Desvenlafaxine (Pristiq), Duloxetine (Cymbalta), Levomilnacipran (Fetzima), Milnacipran (Ixel, Savella), Venlafaxine (Effexor), Vilazodone (Viibryd), Vortioxetine (Trintellix), Nefazodone (Dutonin, Nefadar, Serzone), Trazodone (Desyrel), Reboxetine (Edronax), Teniloxazine (Lucelan, Metatone), Viloxazine (Vivalan), Bupropion (Wellbutrin), Amitriptyline (Elavil, Endep), Amitriptylinoxide (Amioxid, Ambivalon, Equilibrin), Clomipramine (Anafranil), Desipramine (Norpramin, Pertofrane), Dibenzepin (Noveril, Victoril), Dimetacrine (Istonil), Dosulepin (Prothiaden), Doxepin (Adapin, Sinequan), Imipramine (Tofranil), Lofepramine (Lomont, Gamanil), Melitracen (Dixeran, Melixeran, Trausabun), Nitroxazepine (Sintamil), Nortriptyline (Pamelor, Aventyl), Noxiptiline (Agedal, Elronon, Nogedal), Opipramol (Insidon), Pipofezine (Azafen/Azaphen), Protriptyline (Vivactil), Trimipramine (Surmontil), Amoxapine (Asendin), Maprotiline (Ludiomil), Mianserin (Tolvon), Mirtazapine (Remeron), Setiptiline (Tecipul), Isocarboxazid (Marplan), Phenelzine (Nardil), Tranylcypromine (Parnate), Selegiline (Eldepryl, Zelapar, Emsam), Caroxazone (Surodil, Timostenil), Metralindole (Inkazan), Moclobemide (Aurorix, Manerix), Pirlindole (Pirazidol), Toloxatone (Humoryl), Agomelatine (Valdoxan), Esketamine (Spravato), Ketamine (Ketalar), Tandospirone (Sediel), Tianeptine (Stablon, Coaxil), Amisulpride (Solian), Aripiprazole (Abilify), Brexpiprazole (Rexulti), Lurasidone (Latuda), Olanzapine (Zyprexa), Quetiapine (Seroquel), Risperidone (Risperdal), Trifluoperazine (Stelazine), Buspirone (Buspar), Lithium (Eskalith, Lithobid), Modafinil (Provigil), Thyroxine (T4), Triiodothyronine (T3). In an embodiment the further active agent is selected from Celexa (citalopram), Cymbalta (duloxetine), Effexor (venlafaxine), Lexapro (escitalopram), Luvox (fluvoxamine), Paxil (paroxetine), Prozac (fluoxetine), Remeron (mirtazapine), Savella (milnacipran), Trintellix (vortioxetine), Vestra (reboxetine), Viibryd (vilazodone), Wellbutrin (bupropion), Zoloft (sertraline).

### BRIEF DESCRIPTION OF THE DRAWINGS

FIG. 1 is a schematic of a one-step synthesis of 5-MeO-DMT from the reaction of 4-methoxyphenylhydrazine hydrochloride with (N,N)-dimethylamino)butanal dimethyl acetal.

FIG. 2 is a schematic of a three step synthesis of 5-MeO-DMT. The first step involves the reaction of 5-methoxyindole with oxalyl chloride. The resultant product is aminated with dimethylamine and then is reduced with lithium aluminium hydride.

FIG. 3 is a schematic route for the formation of a powder form of 5-MeO-DMT using a spray drying process.

FIG. 4 is an overview of the slug mucosal irritation (SMI) test. (A) First 15 minute contact period between slug and test item. (B) Slug is transferred onto a wet paper towel in a new Petri dish for 1 hour. (C) Second 15 minute contact period between slug and test item. (D) Slug is transferred onto a wet paper towel in a new Petri dish for 1 hour. (E) Third 15 minute contact period between slug and test item.

FIG. 5 is a graph showing that the benzoate salt of 5-MeO-DMT has higher permeation compared with the hydrochloride salt, as per the experiment detailed in Example 7.

FIG. 6 is an XRPD diffractogram of 5-MeO-DMT benzoate prior to particle size reduction.

FIG. 7 is an XRPD diffractogram of 5-MeO-DMT benzoate following particle size reduction.

FIG. 8 is an XRPD diffractogram of FIGS. 6 and 7 overlaid on one another.

FIG. 9 is a graph showing the mean(+/−SD) 5-MeO-DMT (BPL003) plasma linear concentration-time plot.

FIG. 10 is a graph showing the mean(+/−SD) 5-MeO-DMT (BPL003) plasma log concentration-time plot.

FIG. 11 is a graph showing the mean(+/−SD) 5-MeO-DMT (BPL002) plasma linear concentration-time plot.

FIG. 12 is a graph showing the mean(+/−SD) 5-MeO-DMT (BPL002) plasma log concentration-time plot.

FIG. 13 shows Forced Swim Test results, Time Immobile, for 5-MeO-DMT benzoate, vehicle and imipramine.

FIG. 14 shows Forced Swim Test results, Latency to Immobility, for 5-MeO-DMT benzoate, vehicle and imipramine.

FIG. 15 shows a Dynamic Vapour Sorption (DVS) isotherm for 5-MeO-DMT benzoate.

FIG. 16 shows a DVS isotherm of 5-MeO-DMT hydrochloride (lot 20/20/126-FP).

FIG. 17 shows a DVS isotherm of 5-MeO-DMT hydrochloride (lot 20/45/006-FP).

### DETAILED DESCRIPTION

Double-blind, randomized, Phase 1, single ascending dose studies to evaluate the safety, tolerability and pharmacokinetic profile of intranasal 5-MeO-DMT hydrochloride and intranasal 5-MeO-DMT benzoate in healthy subjects have been performed. The results of these studies have surprisingly shown that only the benzoate salt of 5-MeO-DMT exhibits dose-proportional pharmacokinetics. It is desirable that a compound for use in treatment has dose-proportional pharmacokinetics, for example, to facilitate dose and dose regimen adjustment in patients.

There is therefore provided, in an embodiment, a method of treating a condition or disease in a subject, comprising the administration of 5-MeO-DMT benzoate to a subject in need thereof, wherein the 5-MeO-DMT benzoate has dose-proportional pharmacokinetics.

The present disclosure includes the aspects described above and is further illustrated by the following examples. The examples are intended to illustrate the present disclosure without, however, being limiting in nature. It is understood that the present disclosure encompasses additional embodiments consistent with the foregoing description and following examples.

### EXAMPLES

### Example 1: Synthesis of 5-MeO-DMT (the Free Base) in on Step (the Free Base)

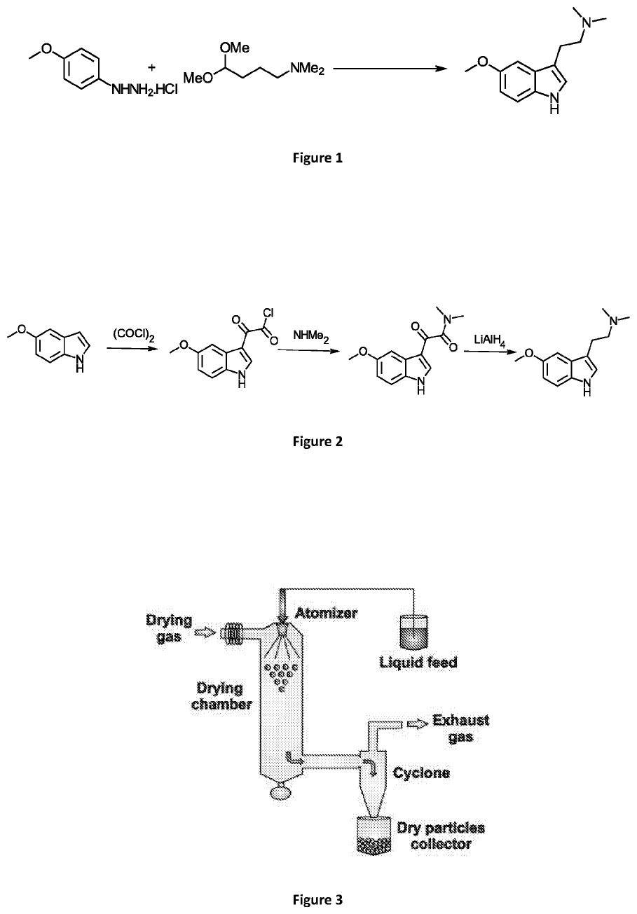

A schematic representation of this reaction is shown in FIG. 1.

Hydrazine (1.0 eq), diethyl acetal (1.2 eq), and aqueous sulfuric acid (0.1 eq) where heated together at 65-75° C. for 18 hours. MTBE (10 vol) was added, followed by adjustment to about pH10 using 12% caustic (about 1.1 eq.). The layers were separated and the aqueous fraction back extracted with MTBE (10 vol). The combined organic fractions were washed with water (10 vol) twice, then evaporated to dryness under vacuum. Yield 100%.

### Example 2: Synthesis of 5-MeO-DMT (the Free Base) in Three Steps

A schematic representation of this reaction is shown in FIG. 2.

Step 1—Add methyl tert-butyl ether (MTBE) (15 vol) into the reaction vessel and cool to −20 to −30° C., before adding oxalyl chloride (1.5 eq), maintaining the temperature at no more than −20° C. Add a solution of 5-methoxyindole (1.0 eq) in THF (1 vol) to the reaction vessel, maintaining the temperature at no more than −20° C. Allow the reaction to warm to 0-5° C. and stir for at least 1 hour, ensuring that no more than 2% of the starting material indole remains.

Cool the reaction to between −20 to −30° C. and add a solution of methanol (1 vol) and MTBE (1 vol), maintaining the temperature at no more than −20° C. Allow the reaction to warm to 0-5° C. over no less than 30 minutes and stir for at least 1 hour.

Filter and wash the solids with MTBE cooled to 0-5° C. Add the washed filtered solids and methanol (20 vol) to a reaction vessel. Heat to 60-65° C. and stir for no more than 30 minutes. Cool to 0-5° C. over no less than 2 hours and stir for no less than 2 hours. Filter and wash the solids with MTBE cooled to 0-5° C. Dry the solids obtained at no more than 40° C. for no less than 12 hours. Yield 95%.

Step 2—Add the compound obtained in step 1 (1.0 eq) to a reaction vessel together with dimethylamine hydrochloride (3.0 eq) and methanol (2 vol). Add 25% NaOMe in methanol (3.5 eq), to the reaction maintaining the temperature at no more than 30° C. Warm to and stir for no less than 5 hours, ensuring that no more than 0.5% of the starting material from step 1 remains. Adjust the temperature to 0-5° C. over no less than 2 hours, then add water (5 vol) over no less than 1 hour with stirring at 0-5° C. for no less than 1 hour.

Filter and wash the solids with water cooled to 0-5° C., and dry the solids obtained at no more than 40° C. for no less than 12 hours. Yield 85%.

Step 3—Add the compound obtained in step 2 (1.0 eq) to a reaction vessel. Add 1M LiAlH4 in THE (1.5 eq) in THF (8 vol) to the reaction maintaining no more than 40° C. Heat at reflux for no less than 4 hours ensuring that no more than 2% of the starting material from step 2 remains.

Adjust to 0-5° C. and add water (0.25 vol) in THE (0.75 vol) over no less than 30 minutes, maintaining no more than 10° C. Then add 15% caustic (0.25 vol) maintaining the temperature at no more than 10° C. Add water (0.65 vol) maintaining the temperature at no more than 10° C. Add THE (0.25 vol) as a vessel rinse and stir the contents at 0-5° C. for no less than 30 minutes. Add sodium sulfate (100 wt %) and stir contents at 0-5° C. for no less than 30 minutes.

Filter and wash the solids with toluene (2×10 vol) and keep liquors separate. Recharge THE liquors to a clean vessel and distil under vacuum to minimum stir. Charge toluene liquors and distil under vacuum to about 10 vol. Then add water (5 vol) and stir for no less than 15 minutes. Stop, settle and remove aqueous layer to waste. Charge with 4% HCl to a pH of between 1-2 (about 4 vol) and stir for no less than 15 minutes. Stop, settle and remove organic layer to waste. Charge MTBE (15 vol). Charge with 15% caustic to a pH between 11-13 (about 0.9 vol). Stir for no less than 15 minutes. Stop, settle and remove aqueous layer to waste. Charge with water (5 vol). Stir for no less than 15 minutes. Stop, settle and remove the aqueous layer to waste.

### Example 3: Synthesis of 5-MeO-DMT Benzoate Salt

5-MeO-DMT (the free base) is dissolved in toluene (1 eq) and benzoic acid (1 eq) in toluene (10 vol) is added over a period of 20 minutes and stirred at room temperature for 2 hours. The resultant precipitation/crystallization was filtered and washed with toluene (2.5 vol) and dried under vacuum at room temperature.

Isopropyl acetate (IPAc) (15.8 vol) was added to the solids obtained above and the temperature was raised to about 73° C. until the solid dissolved. The solution was allowed to cool to 0-5° C. over 2 hours and this temperature was maintained for 1 hour with stirring. The resultant benzoate salt was filtered and vacuum dried at room temperature. Yield 68%.

The benzoate salt of 5-MeO-DMT has improved characteristics over the common hydrochloride salt, including reduced mucosal irritation, increased epithelial permeability and increased stability. 5-MeO-DMT benzoate is a white to off white solid powder, soluble in water at >50 mg/ml with a pH of 7-8 at 50 mg/ml and a pKa of 9.71.

### Example 4: 5-MeO-DMT Powder

A schematic route for the preparation of a powder form of 5-MeO-DMT (or the salt thereof) is shown in FIG. 3. The three main steps in the process are:
1. Spray drying a solution containing the substance(s) of interest (e.g. 5-MeO-DMT, or the salt, thereof inclusive of any excipients). This can be done via an atomizing nozzle such as with rotary atomizers, pressure atomizers, twin fluid nozzles, ultrasonic atomizers, four-fluid nozzles. This is done so as to form droplets capable of generating co-formed particles in the desired particle size range.
2. Drying of the atomized droplets (e.g. with nitrogen gas, optionally at an elevated temperature).
3. Separating and collecting the dried particles from the gas stream (e.g. using a cyclone separator to capture the required size fraction).

In an embodiment, a ProCepT spray dryer is used. In an embodiment, a ProCepT spray dryer with an ultrasonic nozzle is used.

In an embodiment, there is dissolution of 5-MeO-DMT benzoate and HPMC in water to make input solution at a 50:50 ratio.

### Example 5: Slug Mucosal Irritation Assay

The Slug Mucosal Irritation (SMI) assay was initially developed at the Laboratory of Pharmaceutical Technology (UGent) to predict the mucosal irritation potency of pharmaceutical formulations and ingredients. The test utilizes the terrestrial slug Arion lusitanicus. The body wall of the slugs is a mucosal surface composed of different layers. The outer single-layered columnar epithelium that contains cells with cilia, cells with micro-villi and mucus secreting cells covers the subepithelial connective tissue. Slugs that are placed on an irritating substance will produce mucus. Additionally tissue damage can be induced which results in the release of proteins and enzymes from the mucosal surface. Several studies have shown that the SMI assay is a useful tool for evaluating the local tolerance of pharmaceutical formulations and ingredients. A classification prediction model that distinguishes between irritation (mucus production) and tissue damage (release of proteins and enzymes) has been developed. Furthermore, several studies with ophthalmic preparations have shown that an increased mucus production is related to increased incidence of stinging, itching and burning sensations. In 2010 a clinical trial was set up to evaluate the stinging and burning sensations of several diluted shampoos. A 5% shampoo dilution or artificial tears were instilled in the eye and the discomfort was scored by the participants on a 5 point scale during several time points up to 30 min after instillation. The same shampoos were tested in the SMI assay using the Stinging, Itching and Burning (SIB) protocol. This study showed that an increased mucus production was related with an increased incidence of stinging and burning sensations in the human eye irritation test. The relevance of the assay to reliably predict nasal irritation and stinging and burning sensations was demonstrated using several OTC nasal formulations, isotonic, and hypertonic saline.

Furthermore, the test was validated using reference chemicals for eye irritation (ECETOC eye reference data bank). These studies have shown that the SMI assay can be used as an alternative to the in vivo eye irritation tests. Moreover, a multi-center prevalidation study with four participating laboratories showed that the SMI assay is a relevant, easily transferable and reproducible alternative to predict the eye irritation potency of chemicals.

The purpose of this assay was to assess the stinging, itching or burning potential of the test item(s) defined below. Using the objective values obtained for the mucus production the stinging, itching or burning potential of the test item(s) can be estimated by means of the prediction model that is composed of four categories (no, mild, moderate and severe).

### Control Items:

Negative control—Name: Phosphate buffered saline (PBS)
Positive control—Name: 1% (w/v) Benzalkonium chloride in PBS

### Test Items:

### Compound 1

Name: 10% (w/v) Disodium fumarate in PBS
CASRN: 17013-01-3
Batch: KBSJ-PO
Description: colourless solution
Storage condition: room temperature (compounded on the day of the experiment)

### Compound 2

Name: 10% (w/v) Sodium phosphate monobasic in PBS
CASRN: 7558-80-7
Batch: 2A/220991
Description: colourless solution
Storage condition: room temperature (compounded on the day of the experiment)

### Compound 3

Name: 10% (w/v) Sodium acetate in PBS
CASRN: 127-09-3
Batch: 5A/233258
Description: colourless solution
Storage condition: room temperature (compounded on the day of the experiment)

### Compound 4

Name: 10% (w/v) Sodium citrate in PBS
CASRN: 68-04-2
Batch of vial: 5A/241516
Description: colourless solution
Storage condition: room temperature (compounded on the day of the experiment)

Test System: Slugs (Arion lusitonicus); 3 slugs per treatment group. The parental slugs of Arion lusitonicus collected in local gardens along Gent and Aalter (Belgium) are bred in the laboratory in an acclimatized room (18-20° C.). The slugs are housed in plastic containers and fed with lettuce, cucumber, carrots and commercial dog food.

Test Design: A single study was performed. Treatment time was 15 minutes three times on the same day.

### Preparation of Slugs:

Slugs weighing between 3 and 6 g were isolated from the cultures two days before the start of an experiment. The body wall was inspected carefully for evidence of macroscopic injuries. Only slugs with clear tubercles and with a foot surface that shows no evidence of injuries were used for testing purposes. The slugs were placed in a plastic box lined with paper towel moistened with PBS and were kept at 18-20° C. Daily the body wall of the slugs was wetted with 300 μl PBS using a micropipette.

### Test Procedure:

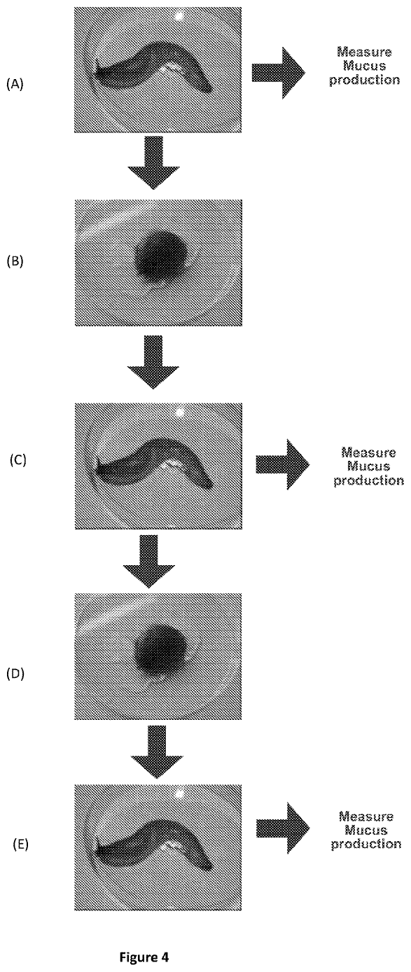

The stinging, itching or burning potency of the test item(s), was evaluated by placing 3 slugs per treatment group 3 times a day on 100 μL of test item in a Petri dish for 15±1 min. After each 15-min contact period the slugs were transferred for 60 min into a fresh Petri dish on paper towel moistened with 1 mL PBS to prevent desiccation. An overview of this can be seen in FIG. 4.

### Mucus Production:

The amount of mucus produced during each contact period was measured by weighing the Petri dishes with the test item before and after each 15-min contact period. The mucus production was expressed as % of the body weight. The slugs were weighed before and after each 15-min contact.

### Classification Prediction Model

Based on the endpoint of the SMI assay the stinging, itching or burning potency of the test item(s) was estimated using a classification prediction model.

The evaluation of the test results was based upon the total amount of mucus production during 3 repeated contact periods with the test item.

For each slug, the mucus production was expressed in % of the body weight by dividing the weight of the mucus produced during each contact period by the body weight of the slug before the start of that contact period. The total mucus was calculated for each slug and then the mean per treatment group was calculated. The classification prediction model shown in the Table below was used to classify the compounds.

### Cut-Off Values for Classification—Potency for Nasal Mucosal Discomfort

| Total Mucus production in % | Stinging, Itching and Burning |
|---|---|
| (mean of n = 3) | (SIB) |
| <5.5% | No |
| ≥5.5 and <10% | Mild |
| ≥10 and <17.5% | Moderate |
| ≥17.5% | Severe |

### Acceptance Criteria

Before a test was considered valid, the following criteria must be met:
the negative control should be classified as causing no stinging, itching and burning (Total mucus production<5.5%)
the positive control item should be classified as causing severe stinging, itching and burning (Total mucus production≥17.5%)

### Irritation Potential

### Amount of Mucus Produced (MP) During Each 15-Min Contact Period (CP) and Total Amount of Mucus Produced

| | MP CP11 | MP CP21 | MP CP31 | Total MP1 | SIB |
|---|---|---|---|---|---|
| Formulation | (%) | (%) | (%) | (%) | Category2 |
| NC-PBS | −0.2 ± 0.3 | −0.6 ± 0.1 | 0.3 ± 0.6 | −0.5 ± 0.7 | No |
| PC-1% BAC | 9.2 ± 1.5 | 8.4 ± 1.2 | 5.9 ± 3.1 | 23.4 ± 3.6 | Severe |
| Disodium fumarate, 10% | 5.0 ± 2.5 | 4.7 ± 1.7 | 3.6 ± 0.8 | 13.3 ± 1.8 | Moderate |
| Sodium phosphate, 10% | 3.3 ± 0.9 | 5.6 ± 0.3 | 6.2 ± 1.3 | 15.2 ± 1.8 | Moderate |
| monobasic | | | | | |
| Sodium acetate, 10% | 3.3 ± 0.2 | 3.9 ± 0.4 | 3.9 ± 0.2 | 11.0 ± 0.8 | Moderate |
| Sodium citrate, 10% | 4.2 ± 0.5 | 4.2 ± 0.3 | 4.1 ± 1.1 | 12.5 ± 1.4 | Moderate |

NC: negative control;
PC: positive control;
BAC: benzalkonium chloride
¹Mean ± SD, n = 3
² No: total MP < 5.5%; Mild: 5.5% ≤ total MP < 10%; Moderate: 10% ≤ total MP < 17.5%; Severe: total MP ≥ 17.5%

The average amount of mucus produced during each 15-min contact period and total mucus production (total MP) is presented in the Table above. According to the classification prediction model of the SMI test, the negative control (untreated slugs) did not induce reactions in the slugs (mean total MP<5.5%). The positive control on the other hand (DDWM/SLS 80/20) induced a high mucus production during each contact period (mean total MP 17.5%) resulting in a classification as severe stinging, itching, and burning (SIB) reactions. The acceptance criteria were met and the experiment was considered valid.

In total, 4 different solutions were tested. The amount of mucus produced during each 15-min contact period was between 10% and 17.5%, indicating moderate SIB reactions. The test items can be ranked according to increasing total mucus production: sodium acetate (10% w/v)<sodium citrate (10% w/v)<disodium fumarate (10% w/v)<sodium phosphate (10% w/v).

### Numerical Data

| | | MP | MP | MP | Total |
|---|---|---|---|---|---|
| Treatment | Replicate | CP1 | CP2 | CP3 | MP |
| NC | 1 | −0.32 | −0.59 | 0.97 | 0.06 |
| | 2 | −0.44 | −0.57 | −0.32 | −1.33 |
| | 3 | 0.14 | −0.70 | 0.35 | −0.21 |
| PC | 1 | 8.08 | 7.91 | 9.29 | 25.28 |
| | 2 | 10.82 | 9.71 | 5.23 | 25.77 |
| | 3 | 8.59 | 7.49 | 3.17 | 19.25 |
| Disodium fumarate, 10% | 1 | 7.83 | 3.56 | 3.14 | 14.53 |
| | 2 | 4.39 | 6.64 | 3.11 | 14.14 |
| | 3 | 2.87 | 3.84 | 4.47 | 11.17 |
| Sodium phosphate, 10% | 1 | 4.33 | 5.34 | 7.41 | 17.07 |
| monobasic | 2 | 2.93 | 5.69 | 6.40 | 15.02 |
| | 3 | 2.74 | 5.83 | 4.89 | 13.46 |
| Sodium acetate, 10% | 1 | 3.47 | 4.24 | 4.10 | 11.80 |
| | 2 | 3.44 | 3.93 | 3.81 | 11.18 |
| | 3 | 3.06 | 3.43 | 3.69 | 10.17 |
| Sodium citrate, 10% | 1 | 4.16 | 4.01 | 3.78 | 11.95 |
| | 2 | 4.75 | 4.03 | 5.33 | 14.12 |
| | 3 | 3.68 | 4.55 | 3.25 | 11.48 |

### Amount of Mucus Produced (MP) During Each 15-Min Contact Period (CP) and Total Amount of Mucus Produced

| | MP CP1¹ | MP CP2¹ | MP CP3¹ | Total MP¹ | SIB |
|---|---|---|---|---|---|
| Formulation | (%) | (%) | (%) | (%) | Category² |
| NC - PBS | −0.2 ± 0.3 | −0.6 ± 0.1 | 0.3 ± 0.6 | −0.5 ± 0.7 | No |
| PC - 1% BAC | 9.2 ± 1.5 | 8.4 ± 1.2 | 5.9 ± 3.1 | 23.4 ± 3.6 | Severe |
| Disodium fumarate, 10% | 5.0 ± 2.5 | 4.7 ± 1.7 | 3.6 ± 0.8 | 13.3 ± 1.8 | Moderate |
| Sodium phosphate, 10% monobasic | 3.3 ± 0.9 | 5.6 ± 0.3 | 6.2 ± 1.3 | 15.2 ± 1.8 | Moderate |
| Sodium acetate, 10% | 3.3 ± 0.2 | 3.9 ± 0.4 | 3.9 ± 0.2 | 11.0 ± 0.8 | Moderate |
| Sodium citrate, 10% | 4.2 ± 0.5 | 4.2 ± 0.3 | 4.1 ± 1.1 | 12.5 ± 1.4 | Moderate |

NC: negative control;
PC: positive control;
BAC: benzalkonium chloride
¹Mean ± SD, n = 3
²No: total MP < 5.5%; Mild: 5.5% ≤ total MP < 10%; Moderate: 10% ≤ total MP < 17.5%; Severe: total MP ≥ 17.5%

### Amount of Mucus Produced (MP) During Each 30-Min Contact Period (CP) and Total Amount of Mucus Produced (Code 00E04)

| Treatment | CP1 30-min | CP2 30-min | Total MP |
|---|---|---|---|
| PBS | −1.0 ± 0.6 | −1.1 ± 0.8 | −2.2 ± 0.6 |
| BAC (1%) | 13.2 ± 4.2 | 18.6 ± 9.8 | 31.8 ± 12.6 |
| Sodium oxalate (1%) | 4.5 ± 1.3 | 6.6 ± 1.0 | 11.1 ± 2.0 |

### Amount of Mucus Produced (MP) During Each 60-Min Contact Period (CP) and Total Amount of Mucus Produced

| | Day 1 | Day 2 | |
|---|---|---|---|
| Treatment | CP1 60-min | CP2 60-min | Total MP |
| PBS | −0.2 ± 0.7 | −0.7 ± 0.5 | −0.9 ± 0.5 |
| BAC (1% CP1 & 3.5% CP2) | 21.9 ± 4.8 | 9.7 ± 3.2 | 31.6 ± 2.5 |
| Sodium oxalate (1% CP1 & | 11.2 ± 3.9 | 16.0 ± 4.0 | 27.1 ± 2.3 |
| 3.5% CP2) | | | |

### Amount of Mucus Produced (MP) During a 60-Min Contact Period (CP)

| Treatment | CP1 60-min | | |
|---|---|---|---|
| PBS | −0.2 | ± | 1.0 |
| BAC (1%) | 15.0 | ± | 1.9 |
| Sodium benzoate (1%) | 2.6 | ± | 0.3 |
| Sodium benzoate (10%) | 6.9 | ± | 1.2 |

### Results

The total MP for a 60-min treatment (historical data) was compared with the total MP of the SIB protocol (3×15-min treatment; current data). In the Table below a ranking is proposed from least SIB reactions to highest SIB reactions:

| | Concen- | | Total MP |
|---|---|---|---|
| Compound | tration | Treatment time | (% body weight) |
| Sodium benzoate | 1% | 60-min | 2.6 |
| Sodium benzoate | 10% | 60-min | 6.9 |
| Sodium acetate | 10% | 45-min (3 × 15-min) | 11.0 |
| Sodium citrate | 10% | 45-min (3 × 15-min) | 12.5 |
| Disodium fumarate | 10% | 45-min (3 × 15-min) | 13.3 |
| Sodium phosphate | 10% | 45-min (3 × 15-min) | 15.2 |
| Sodium oxalate | 1% | 60-min | 11.2 |

Sodium oxalate appears to be the most irritating salt since a 1% concentration results in 11.2% total MP after 1 hour of contact. Sodium benzoate is the least irritating salt.

### Example 6: Further Slug Mucosal Irritation (SMI) Testing

5-MeO-DMT as a freebase compound is known to be highly irritating to the mucosal lining; therefore, it is commonly prepared as a salt for insufflation. The hydrochloride (HCl) salt of 5-MeO-DMT is most commonly used due to ease of crystallisation. However, it is known that the HCl salt of 5-MeO-DMT is still quite irritating to the mucosal lining.

Following the results above indicating that sodium benzoate is the least irritating salt of those studied; further SMI testing was performed on 5-MeO-DMT benzoate and the common 5-MeO-DMT HCl salt according to the previously described methods (of the previous Example). The results of this are shown below:

| | Concentration | Total MP |
|---|---|---|
| Compound | (w/v) | (% body weight) |
| 5-MeO-DMT benzoate | 10% | 7.38 |
| 5-MeO-DMT HCl | 10% | 10.27 |
| Benzylkonium (positive control) | 10% | 17.56 |
| PBS (negative control) | 10% | −0.77 |

The 5-MeO-DMT benzoate produced ‘mild’ irritation compared to the 5-MeO-DMT HCl which scored as ‘moderate’ on testing.

### Example 7: Permeation Data

The use of ovine nasal epithelium to study nasal drug absorption is a technique which is well known to the person skilled in the art.

The permeation of 5-MeO-DMT benzoate and 5-MeO-DMT HCl has been studied by the current applicants. Dosing solutions corresponding to 1.25% concentration were prepared in water and applied to ovine nasal epithelium. The average cumulative (pg/cm²) of permeation of the benzoate and hydrochloride salt are shown in the Table below (mean±SD, n=5):

| Time (min) | | 0.0 | 10.0 | 20.0 | 30.0 | 40.0 | 50.0 | 60.0 | 75.0 | 90.0 |
|---|---|---|---|---|---|---|---|---|---|---|
| Cumulative | 5-MeO-DMT | 0.00 | 0.20 | 3.46 | 9.30 | 15.46 | 21.51 | 27.30 | 33.34 | 39.77 |
| amount | Benzoate | (0.00) | (0.35) | (3.07) | (6.46) | (10.00) | (11.42) | (13.73) | (14.80) | (14.81) |
| (μg/cm² | 5-MeO-DMT | 0.00 | 0.33 | 3.30 | 8.26 | 13.33 | 18.77 | 23.43 | 29.52 | 35.36 |
| (SD)) | Hydrochloride | (0.00) | (0.52) | (3.51) | (6.70) | (8.58) | (10.75) | (11.38) | (12.77) | (13.29) |

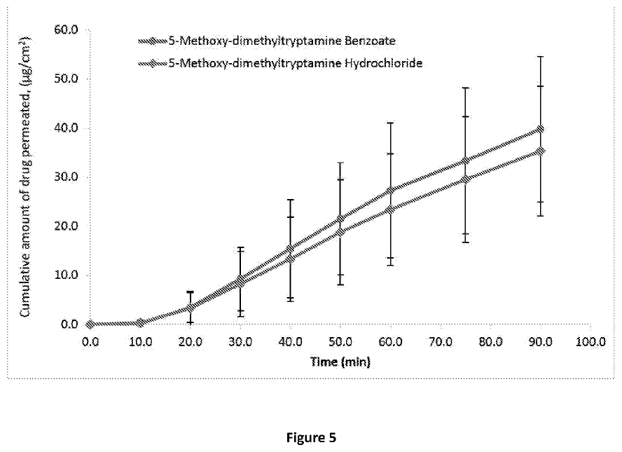

The cumulative amount of 5-MeO-DMT benzoate and 5-MeO-DMT hydrochloride which permeated through ovine nasal epithelium per unit area following application of 1.25% dosing solutions prepared in water (mean±SD, n=5) can be seen in FIG. 5. As can be seen, the benzoate salt has higher permeation across the epithelium. The above data obtained in the above test show that the 5-MeO-DMT benzoate salt gives higher permeation with less mucosal irritation than the commonly used HCl salt; and so this combination of properties makes the benzoate salt an ideal candidate for mucosal delivery. For example, less 5-MeO-DMT benzoate salt may be needed by inhalation to provide the same benefit as the HCl salt and the benzoate salt is less irritating, and so provides a synergistic benefit. Smaller amounts of compound also make inhalation easier to accomplish.

### Example 8: X-Ray Powder Diffraction (XRPD) of 5-MeO-DMT Benzoate

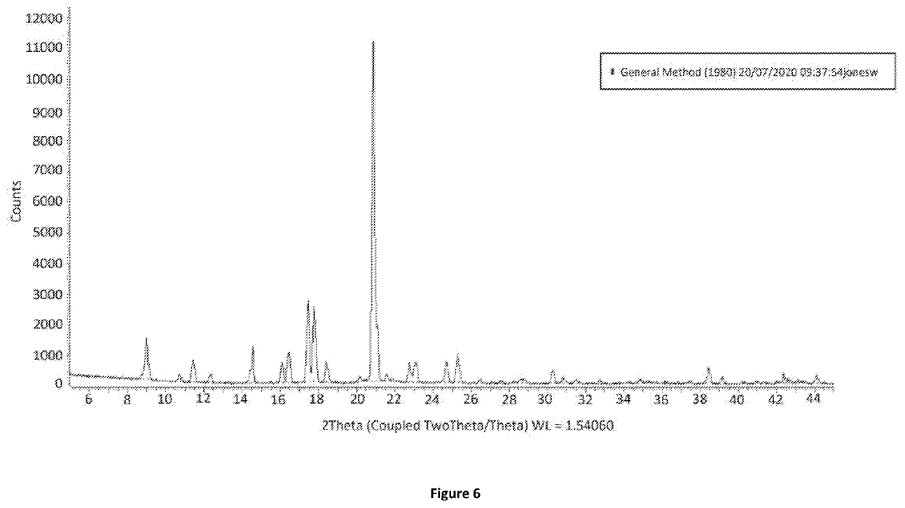

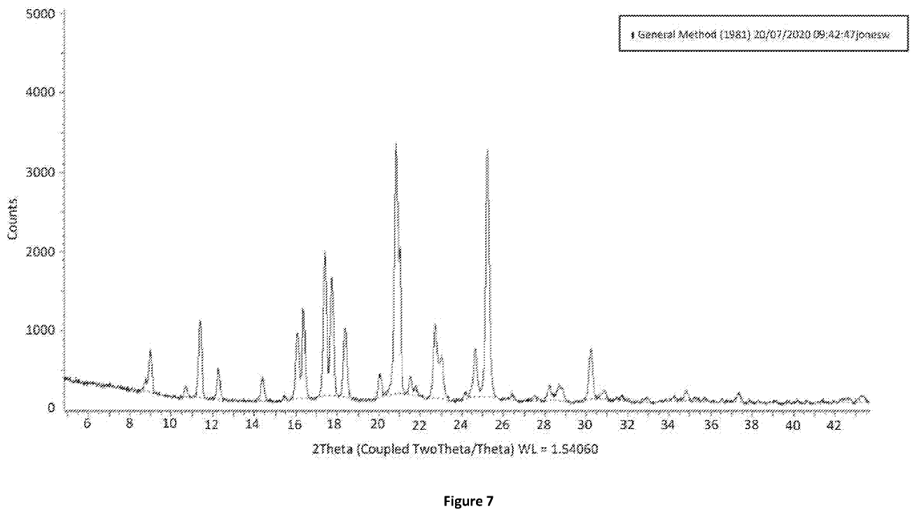

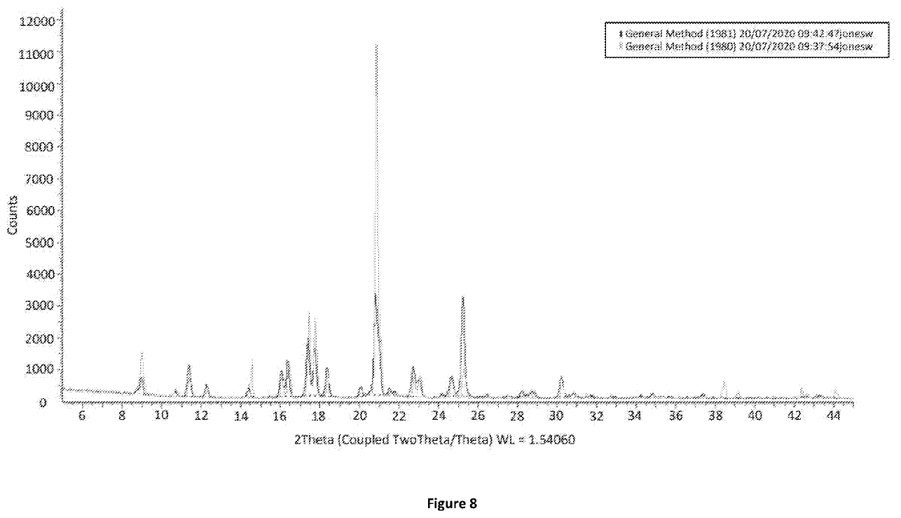

The XRPD pattern of 5-MeO-DMT benzoate salt, was acquired before and following particle size reduction with a mortar and pestle. This reduced the intensity of dominant diffractions and revealed that the XRPD pattern of the benzoate salt was prone to preferred orientation prior to particle size reduction, which is a function of the habit and particle size of the material. XRPD patterns of the benzoate salt prior to and following particle size reduction can be seen in FIGS. 6 and 7 respectively. The XRPD patterns of the benzoate salt prior to and following particle size reduction overlaid on one another can be seen in FIG. 8. In an embodiment, there is provided crystalline 5-MeO-DMT benzoate, characterised by peaks in an XRPD diffractogram at 17.5, 17.7 and 21.0° 2θ±0.1° 2θ. In an embodiment, there is provided crystalline 5-MeO-DMT benzoate, characterised by peaks in an XRPD diffractogram at 17.5, 17.7 and 21.0° 2θ±0.2° 2θ. In an embodiment, there is provided crystalline 5-MeO-DMT benzoate, characterised by peaks in an XRPD diffractogram at 17.5, 17.7 and 21.0° 2θ±0.3° 2θ. In an embodiment, there is provided crystalline 5-MeO-DMT benzoate, characterised by peaks in an XRPD diffractogram at 17.5, 17.7 and 21.0° 2θ±0.1° 20 as measured by x-ray powder diffraction using an x-ray wavelength of 1.5406 Å. In an embodiment, there is provided crystalline 5-MeO-DMT benzoate, characterised by peaks in an XRPD diffractogram at 17.5, 17.7 and 21.0° 2θ±0.2° 20 as measured by x-ray powder diffraction using an x-ray wavelength of 1.5406 Å. In an embodiment, there is provided crystalline 5-MeO-DMT benzoate, characterised by peaks in an XRPD diffractogram at 17.5, 17.7 and 21.0° 2θ±0.3° 2θ as measured by x-ray powder diffraction using an x-ray wavelength of 1.5406 Å. In an embodiment, there is provided crystalline 5-MeO-DMT benzoate, characterised by peaks in an XRPD diffractogram at 17.5, 17.7, 21.0 and 25.3° 2θ±0.1° 2θ. In an embodiment, there is provided crystalline 5-MeO-DMT benzoate, characterised by peaks in an XRPD diffractogram at 17.5, 17.7, 21.0 and 25.3° 2θ±0.2° 2θ. In an embodiment, there is provided crystalline 5-MeO-DMT benzoate, characterised by peaks in an XRPD diffractogram at 17.5, 17.7, 21.0 and 25.3° 2θ±0.3° 2θ. In an embodiment, there is provided crystalline 5-MeO-DMT benzoate, characterised by peaks in an XRPD diffractogram at 17.5, 17.7, 21.0 and 25.3° 2θ±0.1° 2θ as measured by x-ray powder diffraction using an x-ray wavelength of 1.5406 Å. In an embodiment, there is provided crystalline 5-MeO-DMT benzoate, characterised by peaks in an XRPD diffractogram at 17.5, 17.7, 21.0 and 25.3° 2θ±0.2° 2θ as measured by x-ray powder diffraction using an x-ray wavelength of 1.5406 Å. In an embodiment, there is provided crystalline 5-MeO-DMT benzoate, characterised by peaks in an XRPD diffractogram at 17.5, 17.7, 21.0 and 25.3° 2θ±0.3° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.1° 2θ. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.2° 2θ. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.3° 2θ. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.1° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.2° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.5, 17.5, 17.7, 18.5, 21.0, 22.7, 24.7 and 25.3° 2θ±0.3° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 2θ±0.1° 2θ. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 2θ±0.2° 2θ. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 2θ±0.3° 2θ. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 2θ±0.1° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 2θ±0.2° 2θ as measured by x-ray powder diffraction using an x-ray wavelength of 1.5406 Å. In an embodiment, there is provided crystalline 5-MeO-DMT benzoate, characterised by peaks in an XRPD diffractogram at 9.0, 11.5, 14.5, 16.3, 16.5, 17.5, 17.7, 18.5, 21.0, 22.7, 24.7, 25.3 and 30.5° 26±0.3° 2θ as measured by x-ray powder diffraction using an x-ray wavelength of 1.5406 Å. In an embodiment, there is provided crystalline 5-MeO-DMT benzoate, characterised by peaks in an XRPD diffractogram as substantially illustrated in FIGS. 6, 7 or 8.

### Example 9: Spray Drying Parameters

In an embodiment, there is provided a formulation of 5-MeO-DMT benzoate which is a dry powder. In an embodiment, this formulation is presented in a single dose nasal applicator. In an embodiment, 5-MeO-DMT benzoate and HMPC input solutions are made up using sterile water and left to stir until fully dissolved. In an embodiment, the spray drying parameters used to produce a dry powder of 5-MeO-DMT benzoate and HPMC are selected from those set out in the Table below:

| Outlet | Nozzle | Cooling | Airflow | Liquid |
|---|---|---|---|---|
| temperature | air | air | in | feed rate |
| (° C.) | (bar) | (bar) | (m³/min) | (g/min) |
| 80-85 | 1.2 | 0.50 | 0.70 | 2.0 |
| 75-90 | 0.8-1.6 | 0.25-0.75 | 0.50-0.90 | 1.5-2.5 |

### Example 10: a Double-Blind, Randomized. Phase 1, Single Ascending Dose Study to Evaluate the Safety, Tolerability and Pharmacokinetic Profile of Intranasal 5-MeO-DMT Benzoate (BPL-003) in Healthy Subjects

A single ascending dose study to evaluate the safety, tolerability and pharmacokinetic profile of intranasal 5-MeO-DMT benzoate (BPL-003) was performed. The doses tested were 1 mg, 2.5 mg, 4 mg, 6 mg, 8 mg, 10 mg and 12 mg.

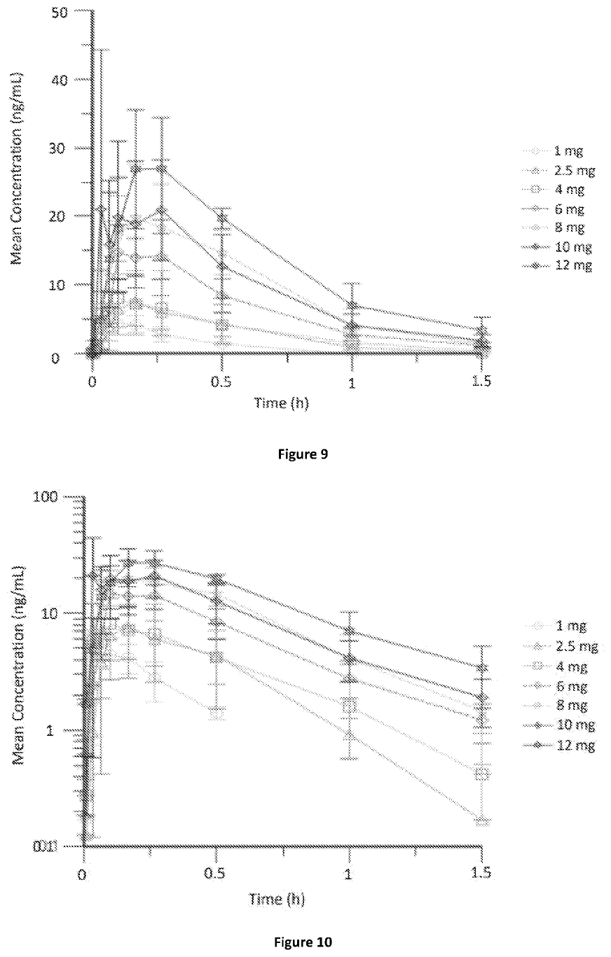

The pharmacokinetics were shown to be approximately dose linear. No dose exceeded the maximum exposure limits defined by previous preclinical work in dogs: Cmax: 421 ng/mL or AUC 220 h·ng/mL. The mean(+/−SD) 5-MeO-DMT plasma linear concentration-time plot and plasma log concentration-time plot are shown in FIGS. 9 and 10, respectively.

The mean Cmax was 29 ng/mL for the 12 mg dosage. The mean Tmax was 9.5 minutes whilst the mean half-life (T_{1/2}) was 21 minutes. Bufotenin, the O-demethylated metabolite of 5-MeO-DMT, was only detected at very low levels at the 6 mg dose level after the 16 minutes timepoint.

Summary statistics for the plasma 5-MeO-DMT, bufotenin PK concentrations, excluding a subject that had a complete profile below the limit of quantification (BLQ) (1 mg), can be seen in the Table below:

| | Treat- | | Time | | | Geometric | | |
|---|---|---|---|---|---|---|---|---|
| | ment | | Point | Mean | SD | Mean | Min | Max |
| Analyte | (mg) | Day | (h) | (ng/mL) | (ng/mL) | (ng/mL) | (ng/mL) | (ng/mL) |
| 5-MeO-DMT | 1.00 | 1 | 0.00 | 0.000 | 0.0000 | | 0.00 | 0.00 |
| | | 1 | 0.01 | 0.1720 | 0.29791 | | 0.00 | 0.516 |
| | | 1 | 0.03 | 2.438 | 1.6918 | 1.81 | 0.513 | 3.69 |
| | | 1 | 0.07 | 5.787 | 1.3352 | 5.68 | 4.44 | 7.11 |
| | | 1 | 0.10 | 4.410 | 1.7099 | 4.19 | 2.78 | 6.19 |
| | | 1 | 0.17 | 4.203 | 1.2027 | 4.08 | 2.86 | 5.18 |
| | | 1 | 0.27 | 2.793 | 1.0527 | 2.65 | 1.70 | 3.80 |
| | | 1 | 0.50 | 1.400 | 0.16643 | 1.39 | 1.28 | 1.59 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 2.50 | 1 | 0.00 | 0.000 | 0.0000 | | 0.00 | 0.00 |
| | | 1 | 0.01 | 0.1265 | 0.25300 | | 0.00 | 0.506 |
| | | 1 | 0.03 | 0.9645 | 0.84420 | | 0.00 | 2.01 |
| | | 1 | 0.07 | 3.848 | 3.4290 | 3.05 | 1.94 | 8.98 |
| | | 1 | 0.10 | 6.495 | 2.5781 | 6.09 | 3.71 | 9.04 |
| | | 1 | 0.17 | 7.608 | 3.5969 | 6.88 | 3.39 | 11.4 |
| | | 1 | 0.27 | 6.005 | 2.5063 | 5.47 | 2.48 | 8.34 |
| | | 1 | 0.50 | 4.365 | 2.8645 | 3.82 | 2.27 | 8.60 |
| | | 1 | 1.00 | 0.9153 | 0.35819 | 0.87 | 0.650 | 1.44 |
| | | 1 | 1.50 | 0.1703 | 0.34050 | | 0.00 | 0.681 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 4.00 | 1 | 0.00 | 0.000 | 0.0000 | | 0.00 | 0.00 |
| | | 1 | 0.01 | 0.2750 | 0.55000 | | 0.00 | 1.10 |
| | | 1 | 0.03 | 5.830 | 6.1749 | | 0.00 | 12.3 |
| | | 1 | 0.07 | 5.517 | 3.6393 | 4.41 | 1.57 | 8.74 |
| | | 1 | 0.10 | 8.057 | 5.3719 | 6.45 | 2.34 | 13.0 |
| | | 1 | 0.17 | 7.203 | 4.4149 | 6.30 | 3.31 | 12.0 |
| | | 1 | 0.27 | 6.655 | 4.0733 | 5.65 | 2.24 | 12.1 |
| | | 1 | 0.50 | 4.188 | 1.7103 | 3.95 | 2.66 | 6.58 |
| | | 1 | 1.00 | 1.597 | 1.0287 | 1.37 | 0.747 | 3.01 |
| | | 1 | 1.50 | 0.4205 | 0.51743 | | 0.00 | 1.06 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 6.00 | 1 | 0.00 | 0.000 | 0.0000 | | 0.00 | 0.00 |
| | | 1 | 0.01 | 0.6150 | 1.2300 | | 0.00 | 2.46 |
| | | 1 | 0.03 | 2.410 | 3.0536 | | 0.00 | 6.84 |
| | | 1 | 0.07 | 6.643 | 2.2793 | 6.25 | 3.26 | 8.22 |
| | | 1 | 0.10 | 14.77 | 3.9577 | 14.44 | 12.0 | 19.3 |
| | | 1 | 0.17 | 14.00 | 2.7221 | 13.81 | 10.9 | 16.0 |
| | | 1 | 0.27 | 14.20 | 3.2512 | 13.95 | 11.0 | 17.5 |
| | | 1 | 0.50 | 8.470 | 2.4380 | 8.25 | 6.51 | 11.2 |
| | | 1 | 1.00 | 2.768 | 0.88526 | 2.65 | 1.63 | 3.78 |
| | | 1 | 1.50 | 1.225 | 0.45041 | 1.17 | 0.861 | 1.84 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 8.00 | 1 | 0.00 | 0.000 | 0.0000 | | 0.00 | 0.00 |
| | | 1 | 0.01 | 0.1230 | 0.27504 | | 0.00 | 0.615 |
| | | 1 | 0.03 | 6.986 | 5.3011 | 5.06 | 1.30 | 14.0 |
| | | 1 | 0.07 | 11.36 | 8.0263 | 9.56 | 4.83 | 24.7 |
| | | 1 | 0.10 | 17.24 | 5.6533 | 16.44 | 9.79 | 24.6 |
| | | 1 | 0.17 | 19.82 | 7.8350 | 18.60 | 11.9 | 30.9 |
| | | 1 | 0.27 | 18.35 | 6.2324 | 17.40 | 9.75 | 25.4 |
| | | 1 | 0.50 | 14.72 | 3.2507 | 14.44 | 11.5 | 18.8 |
| | | 1 | 1.00 | 4.094 | 2.4326 | 3.55 | 2.02 | 6.80 |
| | | 1 | 1.50 | 1.459 | 1.2914 | | 0.00 | 2.87 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 10.00 | 1 | 0.00 | 0.000 | 0.0000 | | 0.00 | 0.00 |
| | | 1 | 0.01 | 0.1840 | 0.41144 | | 0.00 | 0.920 |
| | | 1 | 0.03 | 21.03 | 23.155 | 10.40 | 2.27 | 46.4 |
| | | 1 | 0.07 | 15.95 | 9.2558 | 13.07 | 3.98 | 26.5 |
| | | 1 | 0.10 | 19.89 | 11.010 | 15.99 | 4.27 | 28.6 |
| | | 1 | 0.17 | 18.88 | 9.2412 | 16.29 | 5.72 | 26.7 |
| | | 1 | 0.27 | 20.92 | 7.2947 | 19.73 | 10.4 | 29.7 |
| | | 1 | 0.50 | 12.72 | 4.6551 | 11.99 | 6.98 | 18.2 |
| | | 1 | 1.00 | 4.146 | 1.5827 | 3.93 | 2.47 | 6.77 |
| | | 1 | 1.50 | 1.904 | 0.84052 | 1.72 | 0.719 | 2.86 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 12.00 | 1 | 0.00 | 0.000 | 0.0000 | | 0.00 | 0.00 |
| | | 1 | 0.01 | 1.659 | 3.4080 | | 0.00 | 7.74 |
| | | 1 | 0.03 | 4.806 | 4.2199 | 3.60 | 1.26 | 11.9 |
| | | 1 | 0.07 | 13.76 | 9.7974 | 11.53 | 5.15 | 30.4 |
| | | 1 | 0.10 | 18.24 | 7.3473 | 17.07 | 9.74 | 27.5 |
| | | 1 | 0.17 | 26.88 | 8.7084 | 25.77 | 17.1 | 38.6 |
| | | 1 | 0.27 | 26.95 | 7.4002 | 26.17 | 18.2 | 36.0 |
| | | 1 | 0.50 | 19.75 | 1.5155 | 19.71 | 17.8 | 21.0 |
| | | 1 | 1.00 | 7.052 | 3.1910 | 6.48 | 3.57 | 11.4 |
| | | 1 | 1.50 | 3.416 | 1.8704 | 3.04 | 1.85 | 6.01 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 1.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 2.50 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 4.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 6.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.4867 | 0.84293 | | 0.00 | 1.46 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 8.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 10.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 12.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |

Summary statistics for the derived plasma 5-MeO-DMT, bufotenin PK concentrations, excluding a subject that had a complete profile below the limit of quantification (BLQ) (1 mg), can be seen in the Table below:

| | | Treatment | | | Geometric | | |
|---|---|---|---|---|---|---|---|
| Analyte | Parameter | (mg) | Mean | SD | Mean | Min | Max |
| 5-MeO-DMT | Cmax (ng/mL) | 1.00 | 5.830 | 1.2701 | 5.74 | 4.57 | 7.11 |
| | | 2.50 | 7.925 | 3.3506 | 7.30 | 3.71 | 11.40 |
| | | 4.00 | 8.728 | 4.6996 | 7.60 | 3.31 | 13.00 |
| | | 6.00 | 14.890 | 5.1179 | 14.06 | 7.66 | 19.30 |
| | | 8.00 | 22.140 | 5.5039 | 21.63 | 16.40 | 30.90 |
| | | 10.00 | 32.280 | 14.9572 | 28.55 | 10.40 | 46.40 |
| | | 12.00 | 28.860 | 7.1835 | 28.15 | 20.90 | 38.60 |
| 5-MeO-DMT | Tmax (h) | 1.00 | 0.100 | 0.0577 | 0.09 | 0.07 | 0.17 |
| | | 2.50 | 0.175 | 0.0687 | 0.16 | 0.10 | 0.27 |
| | | 4.00 | 0.117 | 0.0638 | 0.10 | 0.03 | 0.17 |
| | | 6.00 | 0.150 | 0.0882 | 0.13 | 0.07 | 0.27 |
| | | 18.00 | 0.173 | 0.0596 | 0.17 | 0.10 | 0.27 |
| | | 10.00 | 0.140 | 0.1188 | 0.10 | 0.03 | 0.27 |
| | | 12.00 | 0.253 | 0.1445 | 0.23 | 0.17 | 0.50 |
| 5-MeO-DMT | Lambdaz (1/h) | 1.00 | 3.144 | 1.6026 | 2.93 | 2.01 | 4.28 |
| | | 2.50 | 2.278 | 0.7059 | 2.20 | 1.58 | 2.99 |
| | | 4.00 | 2.005 | 0.6177 | 1.93 | 1.31 | 2.61 |
| | | 6.00 | 1.896 | 0.2557 | 1.88 | 1.66 | 2.17 |
| | | 8.00 | 2.492 | 0.6836 | 2.42 | 1.85 | 3.35 |
| | | 10.00 | 2.006 | 0.6505 | 1.92 | 1.17 | 2.91 |
| | | 12.00 | 1.706 | 0.6635 | 1.63 | 1.25 | 2.47 |
| 5-MeO-DMT | t½ (h) | 1.00 | 0.253 | 0.1292 | 0.24 | 0.16 | 0.34 |
| | | 2.50 | 0.326 | 0.1048 | 0.31 | 0.23 | 0.44 |
| | | 4.00 | 0.374 | 0.1225 | 0.36 | 0.27 | 0.53 |
| | | 6.00 | 0.370 | 0.0488 | 0.37 | 0.32 | 0.42 |
| | | 8.00 | 0.295 | 0.0778 | 0.29 | 0.21 | 0.37 |
| | | 10.00 | 0.379 | 0.1337 | 0.36 | 0.24 | 0.59 |
| | | 12.00 | 0.443 | 0.1437 | 0.43 | 0.28 | 0.55 |
| 5-MeO-DMT | AUClast (h*ng/mL) | 1.00 | 1.436 | 0.3956 | 1.40 | 1.01 | 1.79 |
| | | 2.50 | 3.814 | 1.8623 | 3.53 | 2.27 | 6.52 |
| | | 4.00 | 4.507 | 2.1887 | 4.05 | 1.88 | 7.15 |
| | | 6.00 | 8.004 | 2.4941 | 7.66 | 4.62 | 10.57 |
| | | 8.00 | 13.125 | 3.6230 | 12.71 | 9.25 | 17.23 |
| | | 10.00 | 14.349 | 4.5728 | 13.66 | 7.53 | 18.90 |
| | | 12.00 | 18.499 | 3.7803 | 18.19 | 14.45 | 22.33 |
| 5-MeO-DMT | AUCinf (h*ng/mL) | 1.00 | 1.871 | 0.3242 | 1.86 | 1.64 | 2.10 |
| | | 2.50 | 4.388 | 2.1067 | 4.09 | 2.88 | 6.80 |
| | | 4.00 | 5.011 | 2.1449 | 4.61 | 2.38 | 7.39 |
| | | 6.00 | 9.813 | 1.2702 | 9.76 | 8.57 | 11.11 |
| | | 8.00 | 13.977 | 4.1968 | 13.45 | 9.42 | 18.70 |
| | | 10.00 | 15.400 | 4.3753 | 14.86 | 9.37 | 20.41 |
| | | 12.00 | 23.946 | 4.2697 | 23.67 | 19.10 | 27.15 |
| 5-MeO-DMT | Cl/F (L/h) | 1.00 | 542.710 | 94.0402 | 538.62 | 476.21 | 609.21 |
| | | 2.50 | 650.792 | 256.3147 | 611.64 | 367.89 | 867.56 |
| | | 4.00 | 954.076 | 509.4221 | 866.85 | 541.46 | 1682.08 |
| | | 6.00 | 618.346 | 80.0262 | 614.89 | 540.06 | 700.00 |
| | | 8.00 | 618.880 | 195.4794 | 594.78 | 427.81 | 849.14 |
| | | 10.00 | 699.733 | 228.3764 | 672.89 | 489.99 | 1066.94 |
| | | 12.00 | 513.071 | 100.6805 | 506.87 | 441.93 | 628.27 |
| 5-MeO-DMT | Vz/F (L) | 1.00 | 207.161 | 135.5100 | 183.67 | 111.34 | 302.98 |
| | | 2.50 | 317.071 | 204.4948 | 277.60 | 162.61 | 548.98 |
| | | 4.00 | 524.991 | 345.8321 | 449.11 | 222.83 | 1011.75 |
| | | 6.00 | 332.021 | 72.0036 | 326.29 | 248.98 | 377.14 |
| | | 8.00 | 253.099 | 66.2877 | 245.90 | 164.05 | 345.32 |
| | | 10.00 | 407.535 | 287.0370 | 350.66 | 228.42 | 909.63 |
| | | 12.00 | 314.297 | 52.4802 | 311.18 | 254.62 | 353.23 |
| Bufotenine | Cmax (ng/mL) | 1.00 | 0.000 | 0.0000 | | 0.00 | 0.00 |
| | | 2.50 | 0.000 | 0.0000 | | 0.00 | 0.00 |
| | | 4.00 | 0.000 | 0.0000 | | 0.00 | 0.00 |
| | | 6.00 | 0.365 | 0.7300 | | 0.00 | 1.46 |
| | | 8.00 | 0.000 | 0.0000 | | 0.00 | 0.00 |
| | | 10.00 | 0.000 | 0.0000 | | 0.00 | 0.00 |
| | | 12.00 | 0.000 | 0.0000 | | 0.00 | 0.00 |
| Bufotenine | Tmax (h) | 1.00 | 0.000 | 0.0000 | | 0.00 | 0.00 |
| | | 2.50 | 0.000 | 0.0000 | | 0.00 | 0.00 |
| | | 4.00 | 0.000 | 0.0000 | | 0.00 | 0.00 |
| | | 6.00 | 0.067 | 0.1333 | | 0.00 | 0.27 |
| | | 18.00 | 0.000 | 0.0000 | | 0.00 | 0.00 |
| | | 10.00 | 0.000 | 0.0000 | | 0.00 | 0.00 |
| | | 12.00 | 0.000 | 0.0000 | | 0.00 | 0.00 |
| Bufotenine | Lambdaz (1/h) | 1.00 | | | | | |
| | | 2.50 | | | | | |
| | | 4.00 | | | | | |
| | | 6.00 | | | | | |
| | | 8.00 | | | | | |
| | | 10.00 | | | | | |
| | | 12.00 | | | | | |
| Bufotenine | t½ (h) | 1.00 | | | | | |
| | | 2.50 | | | | | |
| | | 4.00 | | | | | |
| | | 6.00 | | | | | |
| | | 8.00 | | | | | |
| | | 10.00 | | | | | |
| | | 12.00 | | | | | |
| Bufotenine | AUClast (h*ng/mL) | 1.00 | | | | | |
| | | 2.50 | | | | | |
| | | 4.00 | | | | | |
| | | 6.00 | 0.073 | | 0.07 | 0.07 | 0.07 |
| | | 8.00 | | | | | |
| | | 10.00 | | | | | |
| | | 12.00 | | | | | |
| Bufotenine | AUCinf (h*ng/mL) | 1.00 | | | | | |
| | | 2.50 | | | | | |
| | | 4.00 | | | | | |
| | | 6.00 | | | | | |
| | | 8.00 | | | | | |
| | | 10.00 | | | | | |
| | | 12.00 | | | | | |

### Example 11: A Double-Blind, Randomized, Phase 1, Single Ascending Dose Study to Evaluate the Safety, Tolerability and Pharmacokinetic Profile of Intranasal 5-MeO-DMT HCl (BPL-002) in Healthy Subjects

A single ascending dose study to evaluate the safety, tolerability and pharmacokinetic profile of intranasal 5-MeO-DMT HCl (BPL-002) was performed. BPL-002 comprises 5-MeO-DMT HCl, HPMC, water for injection (WFI) and a sodium hydroxide solution to adjust pH. An initial stock solution of 0.5% w/w HMPC was prepared using sterile water for injection which was further diluted with sterile water for injection to approximately 90% of the final weight required. The required amount of drug substance (70 mg/ml or 140 mg/ml Freebase) was then dissolved in an aliquot of the 0.5% w/w HPMC stock solution with stirring and the pH of the active stock solution was then adjusted to 6.00+/−0.25 by adding sodium hydroxide (0.1M solution). The IPC measurement was taken to ensure the pH is adjusted within the accepted range before making up to the final weight with sterile water for injection The concentration of the HPMC in the final solution was 0.1% w/w.

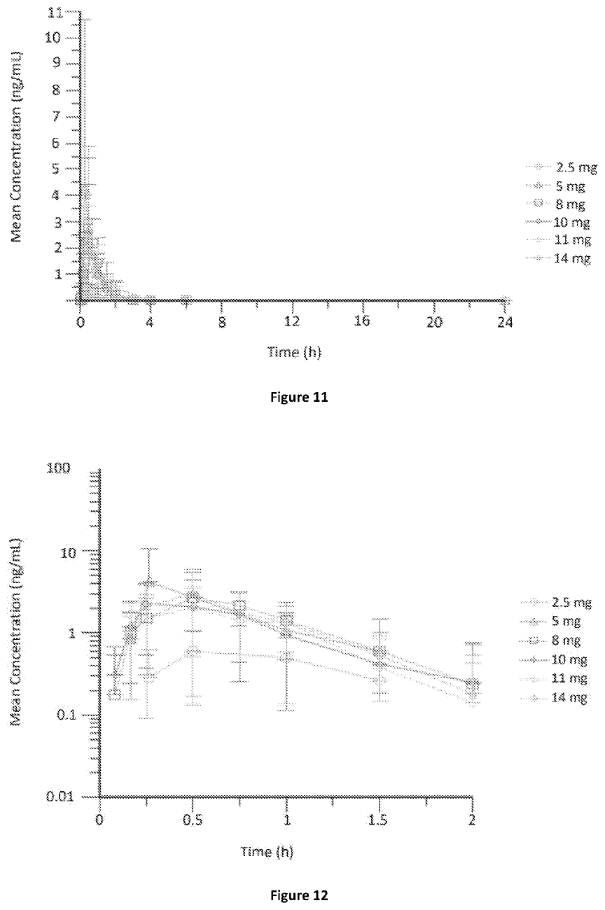

Placebo solutions of 0.1% w/w HPMC were made up by dissolving the required amount of HPMC in sterile water for injection (approximately 90% of final weight) and adjusted for pH 5.75+/−0.25 if necessary by the addition of sodium hydroxide solution (0.05M). The in process check measurement was taken to ensure the pH is adjusted within the accepted range before making up so the final weight with sterile water for injection. The concentration of the HPMC in the final solution was 0.1% w/w. The mean(+/−SD) 5-MeO-DMT plasma linear concentration-time plot and plasma log concentration-time plot are shown in FIGS. 11 and 12, respectively. Summary statistics for the plasma 5-MeO-DMT, bufotenin PK concentrations, can be seen in the Table below:

| | Treat- | | Time | | | Geometric | | |
|---|---|---|---|---|---|---|---|---|
| | ment | | Point | Mean | SD | Mean | Min | Max |
| Analyte | (mg) | Day | (h) | (ng/mL) | (ng/mL) | (ng/mL) | (ng/mL) | (ng/mL) |
| 5-MeO-DMT | 2.50 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.2873 | 0.33535 | | 0.00 | 0.635 |
| | | 1 | 0.50 | 0.6058 | 0.47291 | | 0.00 | 1.14 |
| | | 1 | 1.00 | 0.4973 | 0.35925 | | 0.00 | 0.847 |
| | | 1 | 1.50 | 0.2663 | 0.30826 | | 0.00 | 0.560 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 5.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.8525 | 0.96118 | | 0.00 | 2.23 |
| | | 1 | 0.27 | 4.243 | 6.4483 | 1.86 | 0.541 | 13.9 |
| | | 1 | 0.50 | 2.703 | 2.7488 | 1.95 | 0.830 | 6.79 |
| | | 1 | 1.00 | 1.083 | 0.50356 | 0.99 | 0.508 | 1.65 |
| | | 1 | 1.50 | 0.5968 | 0.40969 | | 0.00 | 0.910 |
| | | 1 | 4.00 | 0.000 | 0.0000 | | 0.00 | 0.00 |
| | | 1 | 24.00 | 0.000 | 0.0000 | | 0.00 | 0.00 |
| 5-MeO-DMT | 8.00 | 1 | 0.00 | 0.000 | 0.0000 | | 0.00 | 0.00 |
| | | 1 | 0.08 | 0.1790 | 0.35800 | | 0.00 | 0.716 |
| | | 1 | 0.17 | 1.016 | 0.76929 | | 0.00 | 1.83 |
| | | 1 | 0.25 | 1.485 | 1.1103 | | 0.00 | 2.61 |
| | | 1 | 0.50 | 2.723 | 1.6869 | 2.42 | 1.58 | 5.22 |
| | | 1 | 0.75 | 2.170 | 0.96161 | 2.05 | 1.59 | 3.28 |
| | | 1 | 1.00 | 1.426 | 0.95805 | 1.22 | 0.608 | 2.48 |
| | | 1 | 1.50 | 0.6000 | 0.84853 | | 0.00 | 1.20 |
| | | 1 | 2.00 | 0.2383 | 0.47650 | | 0.00 | 0.953 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| 5-MeO-DMT | 10.00 | 1 | 0.00 | 0.000 | 0.0000 | | 0.00 | 0.00 |
| | | 1 | 0.08 | 0.3090 | 0.36462 | | 0.00 | 0.710 |
| | | 1 | 0.17 | 1.192 | 1.2090 | | 0.00 | 2.86 |
| | | 1 | 0.25 | 2.282 | 1.7362 | 1.82 | 0.896 | 4.62 |
| | | 1 | 0.50 | 2.100 | 1.5040 | 1.66 | 0.540 | 4.11 |
| | | 1 | 0.75 | 1.680 | 1.4243 | | 0.00 | 3.43 |
| | | 1 | 1.00 | 0.9425 | 0.82971 | | 0.00 | 1.85 |
| | | 1 | 1.50 | 0.4188 | 0.49004 | | 0.00 | 0.935 |
| | | 1 | 2.00 | 0.2500 | 0.50000 | | 0.00 | 1.00 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| 5-MeO-DMT | 11.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 1.040 | 1.2363 | | 0.00 | 2.44 |
| | | 1 | 0.25 | 1.500 | 1.4088 | | 0.00 | 3.03 |
| | | 1 | 0.50 | 2.055 | 1.5511 | | 0.00 | 3.61 |
| | | 1 | 0.75 | 1.428 | 0.97930 | | 0.00 | 2.23 |
| | | 1 | 1.00 | 1.355 | 0.45804 | 1.28 | 0.728 | 1.80 |
| | | 1 | 1.50 | 0.3823 | 0.45258 | | 0.00 | 0.887 |
| | | 1 | 2.00 | 0.1420 | 0.28400 | | 0.00 | 0.568 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| 5-MeO-DMT | 14.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 0.8765 | 0.72168 | | 0.00 | 1.71 |
| | | 1 | 0.25 | 2.089 | 1.7798 | 1.62 | 0.646 | 4.68 |
| | | 1 | 0.50 | 3.030 | 2.8618 | 2.33 | 1.31 | 7.31 |
| | | 1 | 0.75 | 1.761 | 1.3215 | 1.41 | 0.553 | 3.60 |
| | | 1 | 1.00 | 1.354 | 0.77682 | 1.16 | 0.502 | 2.19 |
| | | 1 | 1.50 | 0.5330 | 0.38472 | | 0.00 | 0.865 |
| | | 1 | 2.00 | 0.1808 | 0.36150 | | 0.00 | 0.723 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| Bufotenine | 2.50 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 5.00 | 1 | 0.00 | 0.000 | 0.0000 | | 0.00 | 0.00 |
| | | 1 | 0.01 | 0.000 | 0.0000 | | 0.00 | 0.00 |
| | | 1 | 0.03 | 0.000 | 0.0000 | | 0.00 | 0.00 |
| | | 1 | 0.07 | 0.000 | 0.0000 | | 0.00 | 0.00 |
| | | 1 | 0.10 | 0.000 | 0.0000 | | 0.00 | 0.00 |
| | | 1 | 0.17 | 0.000 | 0.0000 | | 0.00 | 0.00 |
| | | 1 | 0.27 | 0.000 | 0.0000 | | 0.00 | 0.00 |
| | | 1 | 0.50 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 4.00 | 0.000 | 0.0000 | | 0.00 | 0.00 |
| | | 1 | 24.00 | 0.000 | 0.0000 | | 0.00 | 0.00 |
| Bufotenine | 8.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 0.000 | 0.0000 | | 0.00 | 0.00 |
| | | 1 | 0.25 | 0.000 | 0.0000 | | 0.00 | 0.00 |
| | | 1 | 0.50 | 0.000 | 0.0000 | | 0.00 | 0.00 |
| | | 1 | 0.75 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 2.00 | 0.000 | 0.0000 | | 0.00 | 0.00 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| Bufotenine | 10.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 0.000 | 0.0000 | | 0.00 | 0.00 |
| | | 1 | 0.25 | 0.000 | 0.0000 | | 0.00 | 0.00 |
| | | 1 | 0.50 | 0.000 | 0.0000 | | 0.00 | 0.00 |
| | | 1 | 0.75 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 2.00 | 0.000 | 0.0000 | | 0.00 | 0.00 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| Bufotenine | 11.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 0.000 | 0.0000 | | 0.00 | 0.00 |
| | | 1 | 0.25 | 0.000 | 0.0000 | | 0.00 | 0.00 |
| | | 1 | 0.50 | 0.000 | 0.0000 | | 0.00 | 0.00 |
| | | 1 | 0.75 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 2.00 | 0.000 | 0.0000 | | 0.00 | 0.00 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |
| Bufotenine | 14.00 | 1 | 0.00 | 0.000 | 0.0000 | | 0.00 | 0.00 |
| | | 1 | 0.08 | 0.000 | 0.0000 | | 0.00 | 0.00 |
| | | 1 | 0.17 | 0.000 | 0.0000 | | 0.00 | 0.00 |
| | | 1 | 0.25 | 0.000 | 0.0000 | | 0.00 | 0.00 |
| | | 1 | 0.50 | 0.000 | 0.0000 | | 0.00 | 0.00 |
| | | 1 | 0.75 | 0.000 | 0.0000 | | 0.00 | 0.00 |
| | | 1 | 1.00 | 0.000 | 0.0000 | | 0.00 | 0.00 |
| | | 1 | 1.50 | 0.000 | 0.0000 | | 0.00 | 0.00 |
| | | 1 | 2.00 | 0.000 | 0.0000 | | 0.00 | 0.00 |
| | | 1 | 3.00 | 0.000 | 0.0000 | | 0.00 | 0.00 |
| | | 1 | 4.00 | 0.000 | 0.0000 | | 0.00 | 0.00 |
| | | 1 | 6.00 | 0.000 | 0.0000 | | 0.00 | 0.00 |

### Summary Statistics for Derived Plasma 5-MeO-DMT, Bufotenine PK Parameters can be Seen in the Table Below:

| | | Treatment | | | Geometric | | |
|---|---|---|---|---|---|---|---|
| Analyte | Parameter | (mg) | Mean | SD | Mean | Min | Max |
| 5-MeO-DMT | C max (ng/mL) | 2.50 | 0.7630 | 0.26181 | 0.7334 | 0.552 | 1.14 |
| | | 5.00 | 4.486 | 6.28571 | 2.344 | 0.854 | 13.9 |
| | | 8.00 | 2.748 | 1.67032 | 2.450 | 1.58 | 5.22 |
| | | 10.00 | 2.897 | 1.76277 | 2.403 | 0.896 | 4.62 |
| | | 11.00 | 2.453 | 1.04857 | 2.276 | 1.34 | 3.61 |
| | | 14.00 | 3.130 | 2.78770 | 2.486 | 1.66 | 7.31 |
| 5-MeO-DMT | Tmax (h) | 2.50 | 0.6250 | 0.25000 | 0.5946 | 0.500 | 1.00 |
| | | 5.00 | 0.5667 | 0.30912 | 0.5081 | 0.267 | 1.00 |
| | | 8.00 | 0.4375 | 0.12500 | 0.4204 | 0.250 | 0.500 |
| | | 10.00 | 0.4375 | 0.23936 | 0.3913 | 0.250 | 0.750 |
| | | 11.00 | 0.5625 | 0.31458 | 0.5000 | 0.250 | 1.00 |
| | | 14.00 | 0.4167 | 0.16667 | 0.3799 | 0.167 | 0.500 |
| 5-MeO-DMT | t½ (h) | 2.50 | | | | | |
| | | 5.00 | 0.3449 | | 0.3449 | 0.345 | 0.345 |
| | | 8.00 | 0.4917 | 0.25494 | 0.4575 | 0.311 | 0.672 |
| | | 10.00 | 0.4791 | 0.32860 | 0.4190 | 0.247 | 0.711 |
| | | 11.00 | 0.5074 | 0.13230 | 0.4987 | 0.414 | 0.601 |
| | | 14.00 | 0.6213 | 0.26439 | 0.5772 | 0.332 | 0.850 |
| 5-MeO-DMT | Lambdaz (1/h) | 2.50 | | | | | |
| | | 5.00 | 2.010 | | 2.010 | 2.01 | 2.01 |
| | | 8.00 | 1.629 | 0.84432 | 1.515 | 1.03 | 2.23 |
| | | 10.00 | 1.892 | 1.29755 | 1.654 | 0.974 | 2.81 |
| | | 11.00 | 1.414 | 0.36873 | 1.390 | 1.15 | 1.67 |
| | | 14.00 | 1.307 | 0.68493 | 1.201 | 0.816 | 2.09 |
| 5-MeO-DMT | AUClast (h*ng/mL) | 2.50 | 0.5150 | 0.33282 | 0.4247 | 0.150 | 0.957 |
| | | 5.00 | 2.308 | 2.23968 | 1.724 | 0.938 | 5.63 |
| | | 8.00 | 1.860 | 1.77576 | 1.368 | 0.535 | 4.48 |
| | | 10.00 | 1.945 | 1.45260 | 1.376 | 0.277 | 3.82 |
| | | 11.00 | 1.742 | 1.08141 | 1.176 | 0.168 | 2.60 |
| | | 14.00 | 2.256 | 1.57521 | 1.904 | 1.09 | 4.48 |
| 5-MeO-DMT | AUCinf (h*ng/mL) | 2.50 | | | | | |
| | | 5.00 | 6.030 | | 6.030 | 6.03 | 6.03 |
| | | 8.00 | 3.478 | 2.59022 | 2.956 | 1.65 | 5.31 |
| | | 10.00 | 3.399 | 1.74956 | 3.166 | 2.16 | 4.64 |
| | | 11.00 | 2.845 | 0.20333 | 2.842 | 2.70 | 2.99 |
| | | 14.00 | 3.170 | 1.63754 | 2.866 | 1.57 | 4.84 |
| 5-MeO-DMT | Cl/F (L/h) | 2.50 | | | | | |
| | | 5.00 | 829.2 | | 829.2 | 829 | 829 |
| | | 8.00 | 3183 | 2370.68968 | 2706 | 1510 | 4860 |
| | | 10.00 | 3391 | 1745.30775 | 3158 | 2160 | 4630 |
| | | 11.00 | 3876 | 276.97450 | 3871 | 3680 | 4070 |
| | | 14.00 | 5443 | 3117.03619 | 4885 | 2890 | 8920 |
| 5-MeO-DMT | Vz/F (L) | 2.50 | | | | | |
| | | 5.00 | 412.6 | | 412.6 | 413 | 413 |
| | | 8.00 | 1822 | 511.04526 | 1786 | 1460 | 2180 |
| | | 10.00 | 1930 | 401.24315 | 1909 | 1650 | 2210 |
| | | 11.00 | 2864 | 942.54202 | 2785 | 2200 | 3530 |
| | | 14.00 | 5234 | 3705.76364 | 4068 | 1380 | 8780 |
| Bufotenine | C max (ng/mL) | 2.50 | 0.000 | 0.00000 | | 0.00 | 0.00 |
| | | 5.00 | 0.000 | 0.00000 | | 0.00 | 0.00 |
| | | 8.00 | 0.000 | 0.00000 | | 0.00 | 0.00 |
| | | 10.00 | 0.000 | 0.00000 | | 0.00 | 0.00 |
| | | 11.00 | 0.000 | 0.00000 | | 0.00 | 0.00 |
| | | 14.00 | 0.000 | 0.00000 | | 0.00 | 0.00 |
| Bufotenine | Tmax (h) | 2.50 | 0.000 | 0.00000 | | 0.00 | 0.00 |
| | | 5.00 | 0.000 | 0.00000 | | 0.00 | 0.00 |
| | | 8.00 | 0.000 | 0.00000 | | 0.00 | 0.00 |
| | | 10.00 | 0.000 | 0.00000 | | 0.00 | 0.00 |
| | | 11.00 | 0.000 | 0.00000 | | 0.00 | 0.00 |
| | | 14.00 | 0.000 | 0.00000 | | 0.00 | 0.00 |
| Bufotenine | t½ (h) | 2.50 | | | | | |
| | | 5.00 | | | | | |
| | | 8.00 | | | | | |
| | | 10.00 | | | | | |
| | | 11.00 | | | | | |
| | | 14.00 | | | | | |
| Bufotenine | Lambdaz (1/h) | 2.50 | | | | | |
| | | 5.00 | | | | | |
| | | 8.00 | | | | | |
| | | 10.00 | | | | | |
| | | 11.00 | | | | | |
| | | 14.00 | | | | | |
| Bufotenine | AUClast (h*ng/mL) | 2.50 | | | | | |
| | | 5.00 | | | | | |
| | | 8.00 | | | | | |
| | | 10.00 | | | | | |
| | | 11.00 | | | | | |
| | | 14.00 | | | | | |
| Bufotenine | AUCinf (h*ng/mL) | 2.50 | | | | | |

### Example 12: Mouse Forced Swim Test

This study aimed to assess the effect of 5-MeO-DMT Benzoate at three doses in the mouse Forced Swim Test (FST).

### Husbandry

### Housinq and Acclimation

Animals received a 72-hour period of acclimation to the test facility prior to the commencement of testing. Animals were housed four per cage in polycarbonate cages bedded with ¼″ bed-o'cob. Cages were changed, and enrichment provided according to standard operating procedures. Animals were maintained on a 12-hour light/12-hour dark cycle with all experimental activity occurring during the animals' light cycle. All animal use procedures were performed in accordance with the principles of the Canadian Council on Animal Care (CCAC).

### Food and Water

Certified Rodent Diet (LabDiet® 5001) was offered ad libitum. Animals were not fasted prior to, or after the experiment was initiated. Water was provided ad libitum in glass bottles with stainless steel sippers.

### Study Design

### Test Subjects

Male CD-1 mice from Charles River Laboratories (St. Constant, Quebec, Canada) served as test subjects in this study. Animals generally weighed 25-30 g at the time of testing.

### Schedule of Events

| Study Day | Key Event | Procedure |
|---|---|---|
| −8 | Animal arrival | Acclimation to the animal facility |
| −7, to −1 | Daily obs. | Daily health observations |
| 0 | Forced Swim Test | Body weights and observations |
| | | Dosing with 5-MeO DMT Benzoate, |
| | | Imipramine, and vehicle |
| | | Pre-FST behavioural test |
| | | Forced swim test |

### Treatment Groups

Animals were randomly allocated into the following treatment groups:

| | | | Pre- | |
|---|---|---|---|---|
| | | | treatment | Group |
| Group | Treatment | Route | time | Size |
| A | Vehicle | SC | 3 hr | N = 8 |
| B | 5-MeO DMT Benzoate (0.5 | SC | 3 hr | N = 8 |
| | mg/kg) | | | |
| C | 5-MeO DMT Benzoate (1.5 | SC | 3 hr | N = 8 |
| | mg/kg) | | | |
| D | 5-MeO DMT Benzoate (5 mg/kg) | SC | 3 hr | N = 8 |
| E | Imipramine (30 mg/kg) | IP | 3 hr | N = 8 |

### Pre-FST Behavioural Test

On day 0, in addition to the forced swim test animals were evaluated for signs of 5-HT (serotonin) syndrome. Animals were exposed to activity chambers for 10 minutes at two timepoints post dose: (1) 5-15 minutes post dose, and (2) 2.5 hours post dose.

### Forced Swim Test

Male CD-1 mice received the appropriate dose of vehicle, test article, or positive control (treatments summarized above). Following the appropriate pre-treatment time, animals were gently placed into tall glass cylinders filled with water (20-25° C.). After a period of vigorous activity, each mouse adopted a characteristic immobile posture which is readily identifiable. The swim test involves scoring the duration of immobility. Over a 6-minute test session, the latency to first immobility is recorded (in seconds). The duration of immobility (in seconds) during the last 4 minutes of the test is also measured. Activity or inactivity from 0-2 minutes is not recorded.

### Test Articles

### 5-MeODMT Benzoate

BEW: 1.59 (Benzoate salt form)
MW: 340.40 g/mol
Doses: 0.5, 1.5, 5 mg/kg (doses corrected to base)
Route of administration, dose volume: SC., 10 mL/kg
Pre-treatment time: 3 hr
Vehicle: 0.9% Saline

### Imipramine

BEW: 1.13
MW: 280.415 g/mol
Doses: 30 mg/kg (doses corrected to base)
Route of administration, dose volume: IP., 10 mL/kg
Pre-treatment time: 3 hr
Vehicle: 0.9% Saline

### Results

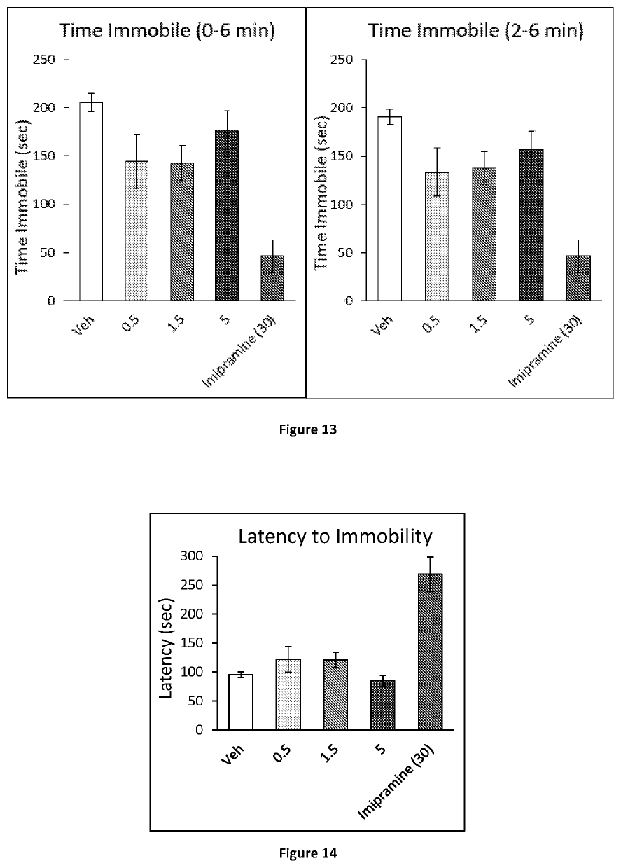

At 3-hour post-dose, over the 6-minute test session, there is a positive trend in reducing the duration of immobility and increasing latency to immobility by the low doses of 5-MeO-DMT benzoate (0.5 and 1.5 mg/kg), compared to vehicle-treated mice (time immobile 2-6 minutes, vehicle: 190.4±7.7 seconds—5-MeO-DMT benzoate: 133.2±24.9 seconds (0.5 mg/kg), 137.6±17.0 seconds (1.5 mg/kg), 156.8±18.7 seconds (5 mg/kg)—Imipramine 46.8±16.6 seconds, FIG. 13. Latency to immobility, vehicle: 95.5±4.6 seconds—5-MeO-DMT benzoate 121.8±22.0 seconds (0.5 mg/kg), 120.9±13.3 seconds (1.5 mg/kg), 85.0±9.5 seconds (5 mg/kg), imipramine 268.6±30.3 second, FIG. 14).

### Example 13: Dynamic Vapour Sorption (DVS) Comparison of 5-MeO-DMT Benzoate and HCl

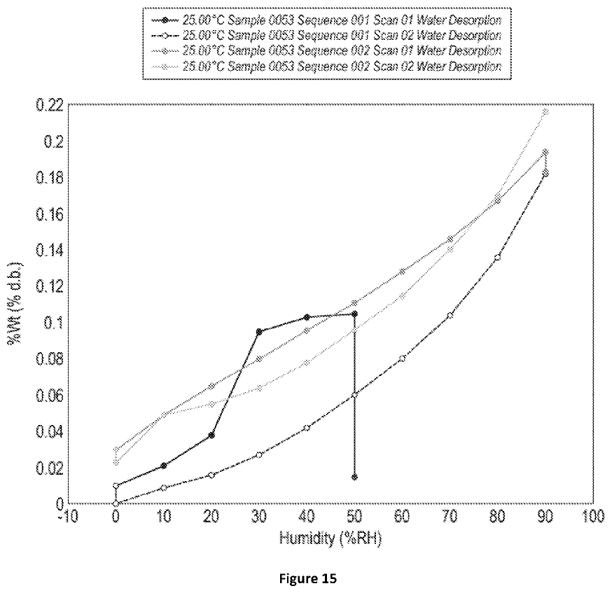

The DVS profile for 5-MeO-DMT benzoate salt, revealed reversible water uptake/loss over the humidity range and no hysteresis. The water uptake/loss from 0 to 90% was gradual and amounted to a maximum of ca 0.20% and was a consequence of wetting of the solid. There was no evidence of form/version modification as a consequence of exposure of 5-MeO-DMT benzoate salt to variable humidity. The DVS isotherm can be seen in FIG. 15.

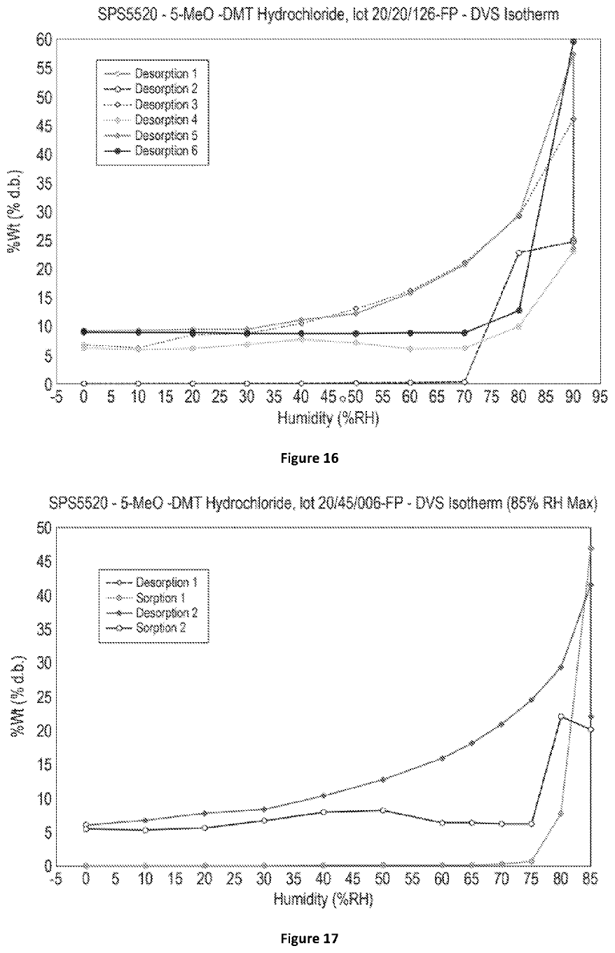

The DVS isotherm of a 5-MeO-DMT Hydrochloride, lot 20/20/126-FP (FIG. 16) was found to undergo significant moisture uptake upon the first sorption cycle from 70% RH. Approximately 23% w/w uptake is observed between 70-80% RH, whereas less than 0.3% w/w moisture uptake from 0-70% RH was observed. A further 20% w/w moisture uptake is observed up to and when held at 90% RH before commencement of the second desorption cycle. Subsequent sorption and desorption cycles follow a similar profile with some observed hysteresis between operations that do not match the original desorption step. These return to ca. 6-9% w/w above the minimum mass recorded at 0% RH, which indicates significant retention of moisture. Upon completion of the DVS cycle, the input material was noted to have completed deliquesced. A modified DVS isotherm of lot 20/45/006-FP (the same crystalline version) was undertaken to examine material behaviour from 60% RH and above. A 2 cycle DVS with desorption beginning from 40-0% RH with sorption from 0-60% RH in 10% RH intervals, followed by incremental 5% RH increases to 65, 70, 75, 80 and finally 85% RH. This was to obtain in-depth profiling of the material towards humidity at these elevated levels. No significant moisture uptake/loss in first desorption-sorption profile between 0-70% RH was noted (FIG. 17) followed by a ca. 0.46% w/w increase from 70-75% RH. A further ca. 7% uptake is observed from 75-80% RH, then ca. 40% from 80-85% w/w. Complete deliquescence of the solids was observed upon isolation of the material post DVS analysis, which had likely occurred above 80% RH. Temperature and humidity are important factors in the processing and storage of pharmaceuticals. DVS provides a versatile and sensitive technique for evaluating the stability of pharmaceutical formulations. The DVS profiles show that the stability of the benzoate salt of 5-MeO-DMT is significantly higher than that of the hydrochloride salt and is therefore a more promising salt for development as a pharmaceutical composition.

### Example 14: Phase 1 Study Results on the Effects of 5-MeO-DMT Benzoate (BPL-003) on Facial Emotion Processing in Psychedelic-Naïve Healthy Subjects

Up to 60% of patients with major depressive disorder (MDD) remain inadequately treated, with approximately 30% of patients with MDD experiencing treatment resistant depression (TRD). Preliminary epidemiological studies of short-lasting psychedelic compounds such as 5-MeO-DMT have reported beneficial outcomes in psychiatric disorders, including alleviation of depressive symptoms. The Facial Expression Recognition Task (FERT) has been shown to be sensitive to changes in positive and negative bias that occur following treatment with antidepressants in healthy volunteers and in patients. To determine changes in positive or negative bias, the FERT was included in a double-blind, placebo-controlled, randomised, single-centre, phase 1 study evaluating the effects of a single ascending intranasal doses of 5-MeO-DMT benzoate (BPL-003).

### Study Population

44 psychedelic naïve healthy participants were enrolled across seven cohorts (see Table 1).

Participants received a single intranasal dose of BPL-003 or matching placebo together with psychological support.

| | Dose | Number of participants | | | Sex | Age |
|---|---|---|---|---|---|---|
| Cohort | (mg) | Placebo | BPL-003 | Total | (male) | (mean ± SE) |
| 1 | 1.0 | 2 | 4 | 6 | 6 | 34.83 ± 3.26 |
| 2 | 2.5 | 2 | 4 | 6 | 3 | 35.67 ± 4.45 |
| 3 | 4.0 | 2 | 4 | 6 | 5 | 29.67 ± 1.26 |
| 4 | 6.0 | 2 | 4 | 6 | 6 | 35.83 ± 2.83 |
| 5 | 8.0 | 2 | 5 | 7 | 5 | 36.57 ± 3.26 |
| 6 | 10.0 | 2 | 5 | 7 | 5 | 36.71 ± 3.46 |
| 7 | 12.0 | 1 | 5 | 6 | 2 | 33.17 ± 4.11 |

### Assessments

The FERT is a digital task in which participants are serially presented faces expressing different emotions (anger, disgust, fear, happiness, sadness, surprise, neutral) at valenced intensities and they respond by selecting the emotion they identify via a touchscreen. The FERT was conducted on Day 1 pre-dose (baseline), on Day 2 at 22-24 hours post-dose, and at follow-up on Day 8. Other assessments of safety, pharmacokinetics, and pharmacodynamics (including questionnaires for subjective effects) were also conducted.

### Results

Several trends were observed indicating that BPL-003 has positive effects on emotional biases. There was a trend showing that participants had an improvement from baseline in recognising happiness when administered 10 mg BPL-003 (Mdn=17.57%, IQR=5.66%−19.17%) and 12 mg BPL-003 (Mdn=7.37%, IQR=6.14%−8.65%) compared to placebo (Mdn=2.5%, IQR=−3.64%−10.26%) on Day 2. On Day 2 there was a trend for the recognition of sadness to decrease in drug-treated (Mdn range=−12.83%−−6.99%) compared to placebo-treated participants (Mdn=−5.27%, IQR=−12.82%-0%).

Improvements in the recognition of happiness can suggest an increase in the bias towards that emotion. Similarly, reductions in the accuracy for recognising sadness suggests a decrease in the bias towards negative emotions. These results show that BPL-003 has a positive effect on emotional biases 22-24 hours after administration, which may provide an early objective measure of improvements in mood.

There is therefore provided a method of increasing a patient's ability to recognise happiness comprising the administration of 5-MeO-DMT, or a composition thereof, optionally as described herein, to a patient in need thereof. There is therefore provided a method of decreasing a patient's ability to recognise sadness comprising the administration of 5-MeO-DMT, or a composition thereof, optionally as described herein, to a patient in need thereof. In an embodiment, it is the benzoate salt of 5-MeO-DMT. In an embodiment, there is provided 5-MeO-DMT, or a composition thereof, for use in one or more of the aforementioned methods. In an embodiment, it is the benzoate salt of 5-MeO-DMT.

What is claimed is:

1. A dose-proportional pharmaceutically acceptable dry powder intranasal composition comprising 1 to 12 mg of 5-methoxy-N,N-dimethyltryptamine (5-MeO-DMT) benzoate dry powder particles, and one or more pharmaceutically acceptable carriers or excipients, for use in a method of treatment, wherein the composition has dose-proportional pharmacokinetics and the method of treatment is selected from treatment of: mood disorders, depression, treatment-resistant forms of depression, addictive disorders, alcohol use disorder or anxiety disorders in a subject in need thereof.

2. The composition for use of claim 1, wherein administration of the composition produces in the subject a Tmax of about 0.05 h to about 0.5 h.

3. The composition for use of claim 1, wherein administration of the composition produces in the subject a mean half-life T½ of about 0.1 h to about 0.55 h.

4. The composition for use of claim 1, wherein administration of the composition produces in the subject a AUClast of about 1.0 h*ng/mL to about 22 h*ng/mL.

5. The composition for use of claim 1, wherein administration of the composition produces in the subject a AUCinf of about 1.5 h*ng/mL to about 27 h*ng/mL.

6. The composition for use of claim any one of claims 1 to 5, wherein the composition comprises 1 to 12 mg of the 5-MeO-DMT benzoate.

7. The composition for use of claim 6, wherein the composition comprises 10 mg of 5-MeO-DMT benzoate, and wherein intranasal administration of the composition produces in the subject one or more of: a Cmax of about 10.4 ng/mL to about 46.4 ng/mL; a Tmax of about 0.03 h to about 0.27 h; a T½ of about 0.24 h to about 0.59 h; a AUClast of about 9.37 h*ng/mL to about 20.41 h*ng/mL; or a AUCinf of about 9.42 h*ng/mL to about 18.7 h*ng/mL.

8. The composition for use of claim 6, wherein the composition comprises 12 mg of 5-MeO-DMT benzoate, wherein intranasal administration of the composition produces in the subject one or more of: a Cmax of about 20.9 ng/mL to about 39 ng/mL; a Tmax of about 0.17 hours to about 0.5 hours; a T½ of about 0.28 h to about 0.55 h; a AUClast of about 14.45 h*ng/mL to about 22 h*ng/mL; or a AUCinf of about 19.10 h*ng/mL to about 27 h*ng/mL.

9. The composition for use of any one of claims 1 to 8, wherein the dry powder particles of 5-MeO-DMT benzoate are characterised by one or more of: particles having a median diameter of less than 2000 μm; particles having a median diameter of less than 15 μm; particles having a median diameter of less than 9 μm; particles having a median diameter of greater than 500 μm; or a particle size distribution having a d10 value of 20-60 μm, a d50 value of 80-120 m, and/or a d90 value of 130-300 μm.

10. The composition for use of claim 9, wherein the 5-MeO-DMT benzoate is present as crystalline 5-MeO-DMT benzoate as characterised by one or more peaks in an X-ray powder diffraction (XRPD) diffractogram at 26 values of 17.5°+0.2°, 17.7°±0.20, and 21.0°±0.20.

11. The composition for use of claim 9 for use in a method of treatment of a mood disorder.

12. The composition for use of claim 10 for use in a method of treatment of a mood disorder.

13. The composition for use of claim 9 for use in a method of treatment of depression.

14. The composition for use of claim 10 for use in a method of treatment of depression.

15. The composition for use of claim 9 for use in a method of treatment of treatment resistant depression.

16. The composition for use of claim 10 for use in a method of treatment of treatment resistant depression.

17. The composition for use of claim 9 for use in a method of treatment of anxiety.

18. The composition for use of claim 10 for use in a method of treatment of anxiety.

19. The composition for use of claim 9 for use in a method of treatment of alcohol use disorder.

20. The composition for use of claim 10 for use in a method of treatment of alcohol use disorder.

21. An intranasal delivery device comprising a dose-proportional pharmaceutically acceptable dry powder intranasal composition comprising 8 to 12 mg of 5-MeO-DMT benzoate dry powder particles, and one or more pharmaceutically acceptable carriers or excipients, for use in a method of treatment, wherein the composition has dose-proportional pharmacokinetics and the method of treatment of depression.

22. An intranasal delivery system comprising an intranasal delivery device comprising a dose-proportional pharmaceutically acceptable dry powder intranasal composition comprising 8 to 12 mg of 5-MeO-DMT benzoate dry powder particles, and one or more pharmaceutically acceptable carriers or excipients, for use in a method of treatment, wherein the composition has dose-proportional pharmacokinetics and the method of treatment of depression.

23. The intranasal delivery system for use of claim 22, further comprising instructions for use.

24. The intranasal delivery device for use of claim 21, wherein the device is single use.

25. The intranasal delivery device for use of claim 21, wherein the device comprises a single dose of 5-MeO-DMT.

26. A dose-proportional pharmaceutically acceptable dry powder intranasal composition comprising 10 mg of 5-methoxy-N,N-dimethyltryptamine (5-MeO-DMT), or a pharmaceutically acceptable salt thereof, dry powder particles, and one or more pharmaceutically acceptable carriers or excipients, for use in a method of increasing a patient's ability to recognise happiness.

27. A dose-proportional pharmaceutically acceptable dry powder intranasal composition comprising 10 mg of 5-methoxy-N,N-dimethyltryptamine (5-MeO-DMT), or a pharmaceutically acceptable salt thereof, dry powder particles, and one or more pharmaceutically acceptable carriers or excipients, for use in a method of decreasing a patient's ability to recognise sadness.
